(19) United States
(12) Patent Application Publication
(10) Pub. No.: US 2010/0254961 A1
(43) Pub. Date: Oct. 7, 2010

(54) WATER-SOLUBLE ELECTROSPUN SHEET

(75) Inventors: Toshihiko Nishio, Mie-Prefecture (JP); Hidetoshi Sugino, Mie-Prefecture (JP); Tsutomu Okubo, Mie-Prefecture (JP); Shoichi Ishigaki, Mie-Prefecture (JP)

Correspondence Address: BURR & BROWN, PO BOX 7068, SYRACUSE, NY, 13261-7068, US

(73) Assignee: Taiyokagaku Co., Ltd., Yokkaichi-shi (JP)

(21) Appl. No.: 12/676,030

(22) PCT Filed: Sep. 4, 2008
(86) PCT No.: PCT/JP2008/065987

§ 371 Date: Jun. 16, 2010

(30) Foreign Application Priority Data
Sep. 5, 2007 (JP) 2007-230518
Dec. 25, 2007 (JP) 2007-331778

(51) Int. Cl. A61K 38/43; A61K 47/00; A61K 47/38; A61K 47/42; A61K 47/30; A61K 31/715; A61K 35/54; A61K 36/82; A61K 36/48

(52) U.S. Cl. 424/94.1; 514/773; 514/781; 514/783; 514/772.3; 514/54; 424/581; 424/729; 424/757

(57) ABSTRACT

A water-soluble electrospun sheet, containing a water-soluble base material made of at least one material selected from a group consisting of: high-molecular proteins and decomposition products thereof; cellulose-based polymers; plant-based polymers and decomposition products thereof; vinyl-based polymers; acrylic-based polymers; and water-soluble polysaccharides; is provided. In addition to the water-soluble base material, the sheet may further contain at least one functional component selected from among: emulsifying components; stabilizing components; antimicrobial components; humectant components; skin-whitening components; anti-ultraviolet components; astringent components; keratin-softening components; anti-inflammatory components; and coloring components.

### FIELD OF THE INVENTION

The present invention relates to a water-soluble electrospun sheet.

### BACKGROUND ART

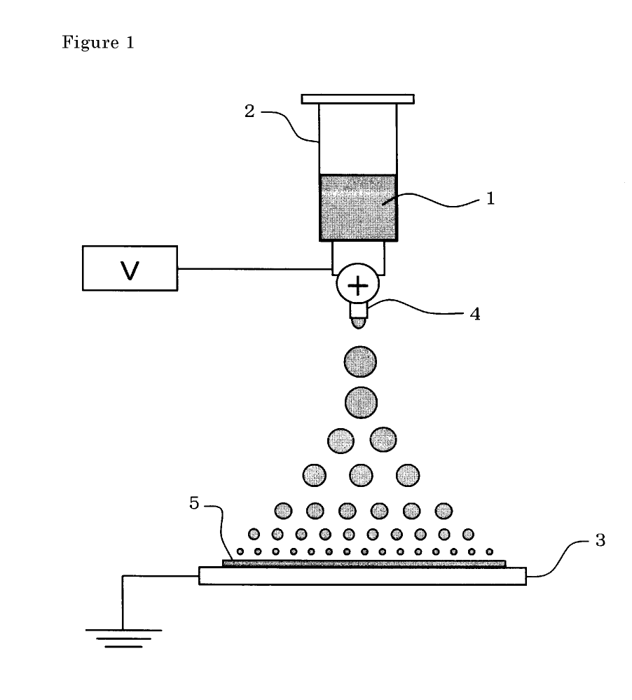

Arts of manufacturing polymer fibers of nanometer size by an electrospinning method have been known from before (for example, Patent Document 1). Described briefly, this method is as follows. A container 2, storing a base material solution 1, which is to be a raw material, and a target electrode 3 are disposed as shown in FIG. 1. A nozzle 4 capable of ejecting the base material solution 1 is disposed at a tip of the container 2. Here, when the base material solution 1 is ejected from the nozzle 4 in a state where a high voltage is applied across the nozzle 4 and the target electrode 3, the base material solution 1 is formed into filamentous fibers along electrical lines of force as it moves from the nozzle 4 to the target electrode 3, and fibers 5 are thereby formed on the target electrode 3.

This method has a characteristic of enabling forming of fibers in the order of 10 nm to several 10 μm and forming of sheets or mats by assembly of the fibers. The fibers manufactured by this method are thin in diameter, the sheets or mats that are the assemblies thereof are high in porosity, and thus wide applications to diverse usages are being considered. The fiber assembly has a fine internal structure and surface structure, is large in specific surface area, and exhibits excellent characteristics in usage, for example, as an adsorbent.
Patent Document 1: U.S. Pat. No. 6,656,394

### DISCLOSURE OF THE INVENTION

### Object(s) of the Invention

However, due to research on the electrospinning method being short in history, the circumstances are such that the method can hardly be said to be adequately developed in regard to application examples. Also thus far, sheets obtained by the electrospinning method are insoluble in water and many of the applications are applications as filters.

The present invention has been made in view of the above circumstances, and an object thereof is to provide a water-soluble electrospun sheet and particularly to provide this sheet as a material for medical or cosmetic applications, etc.

### SUMMARY OF THE INVENTION

The present invention for achieving the above object provides the following:
[1] A water-soluble electrospun sheet containing a water-soluble base material.
[2] The water-soluble electrospun sheet according to [1] with which the water-soluble base material is at least one material selected from a group consisting of: high-molecular proteins and decomposition products thereof; cellulose-based polymers; plant-based polymers and decomposition products thereof; vinyl-based polymers; acrylic-based polymers; and water-soluble polysaccharides.
[3] The water-soluble electrospun sheet according to [1] or [2] with which the water-soluble base material is at least one material selected from a group consisting of: collagen peptide; gelatin, silk fibroin; hydroxypropyl cellulose; quince seed gum; hyaluronic acid; polyvinyl alcohol; sodium polyacrylate; and water-soluble chitosan.
[4] The water-soluble electrospun sheet according to any one of [1] to [3] further containing at least one functional component selected from among: emulsifying components; stabilizing components; antimicrobial components; humectant components; skin-whitening components; anti-ultraviolet components; astringent components; keratin-softening components; anti-inflammatory components; emollient components; and coloring components.
[5] The water-soluble electrospun sheet according to [4] with which the functional component is at least one component selected from a group consisting of: theanine; hyaluronic acid; vitamin C; CoQ10; urea; hydrolyzed eggshell membrane; sodium chondroitin sulfate; glycol salicylate; diphenhydramine hydrochloride; salicylic acid; arbutin; citric acid; succinic acid; tea leaf extract; licorice extract; glycolic acid; allantoin; glycerin; 1,3-butylene glycol; ellagic acid; 2,4-dihydroxybenzophenone; titanium oxide; cerium oxide; and sulfur.
[6] The water-soluble electrospun sheet according to any one of [1] to [5] where the water-soluble electrospun sheet is a cosmetic sheet.
[7] The water-soluble electrospun sheet according to [6] where the cosmetic sheet is a cosmetic facial mask, a cosmetic toner, or a beauty serum.
[8] The water-soluble electrospun sheet according to any one of [1] to [5] where the water-soluble electrospun sheet is a medical sheet.
[9] The water-soluble electrospun sheet according to [8] where the medical sheet contains an antimicrobial substance or an anti-inflammatory substance.

### Effect(s) of the Invention

By the present invention, a water-soluble electrospun sheet can be provided using a predetermined base material. This sheet dissolves in water readily and can thus be used as various materials, such as medical sheets, cosmetic sheets (including cosmetic facial masks, cosmetic toners, and beauty serums), etc. Also by making another functional component (for example, a humectant component, skin-whitening component, anti-ultraviolet component, astringent component, keratin-softening component, anti-inflammatory component, coloring component, etc.,) be contained in addition to the base material, a specific function can be exhibited at a portion at which the sheet is adhered.

### BEST MODE(S) FOR CARRYING OUT THE INVENTION

Although embodiments of the present invention shall now be described with reference to figures and tables, the technical scope of the present invention is not restricted by these embodiments and the present invention can be put into practice in various modes without changing the gist of the invention. Also, the technical scope of the present invention encompasses the scope of equivalence.

A water-soluble base material refers to a material that can be processed to a sheet by an electrospinning method, and examples include: high-molecular proteins and decomposition products thereof; cellulose-based polymers; plant-based polymers and decomposition products thereof; vinyl-based polymers; acrylic-based polymers; and water-soluble polysaccharides. Water-soluble base materials include: materials that readily dissolve in water at a stage before processing to a sheet (for example, collagen peptide, etc.); and materials that do not dissolve readily but become improved in water solubility by being processed to a sheet (for example, gelatin, etc.). In the present invention, a water-soluble base material of either property can be used.

A high-molecular protein refers to a high-molecular protein obtained from an animal, a plant, or a microorganism. If the high-molecular protein itself is water-soluble, it can be subject to the electrospinning method as it is. In a case where a high-molecular protein is not water-soluble or is low in water solubility, it can be subject to an appropriate treatment (for example, an acid treatment, alkali treatment, enzyme treatment, or heat treatment) and used in the present invention as a decomposition product of appropriate size. Examples of high-molecular proteins include collage, casein, albumin, gelatin, silk fibroin, etc. Among these, collagen is a main protein component that makes up connective tissues of animals and takes up approximately 30% of total proteins of the body in a human. Although many types of collagens are known, all have extremely high molecular weights and cannot be dissolved as it is in water. In the present invention, not collagen itself but a collagen peptide that has been made low in molecular weight by hydrolysis, etc., (and preferably having an average molecular weight of approximately 5,000) is preferably used.

A cellulose-based polymer refers to a polymer made up of cellulose or a derivative thereof as units, and examples include methyl cellulose, nitrocellulose, ethyl cellulose, methyl hydroxypropyl cellulose, hydroxypropyl cellulose, hydroxyethyl cellulose, sodium cellulose sulfate, hydroxymethyl cellulose, sodium carboxymethyl cellulose, crystalline cellulose, cellulose powder, etc.

A plant-based polymer refers to a polymer obtained from a plant, and examples include gum arabic, tragacanth gum, galactan, guar gum, carob gum, karaya gum, carrageenan, pectin, agar, quince seed gum, tamarind gum, algae colloid, starch, glycyrrhizin, etc. Although if a plant-based polymer is water-soluble, it can be subject to the electrospinning method as it is, if the plant-based polymer lacks (or is low in) water solubility, it can be subject to an appropriate treatment (for example, an acid treatment, alkali treatment, enzyme treatment, or heat treatment) and used in the present invention as a decomposition product of appropriate size.

A vinyl-based polymer refers to a polymer having a vinyl structure, and examples include polyvinyl alcohol, polyvinyl methyl ether, polyvinyl pyrrolidone, carboxyvinyl polymer, alkyl acrylate/methacrylate copolymer, etc.

Examples of acrylic-based polymers include sodium polyacrylate, polyethyl acrylate, polyacrylamide, etc.

A water-soluble polysaccharide refers to a polysaccharide that is water-soluble, and examples include: starch and hydrolysates thereof; amylose and hydrolysates thereof; amylopectin and hydrolysates thereof; glycogen and hydrolysates thereof; cellulose and hydrolysates thereof; chitin and hydrolysates thereof; agarose and hydrolysates thereof; carrageenan and hydrolysates thereof; heparin and hydrolysates thereof; hyaluronic acid and hydrolysates thereof; pectin and hydrolysates thereof; xyloglucan and hydrolysates thereof; chitosan and hydrolysates thereof; etc.

An emulsifying component refers to a component for mixing an oil and water, and examples include sodium N-alkyloyl methyl taurate, PPG-28-Buteth-35, PEG-2 hydrogenated castor oil, chitosan derivatives, quaternium-18, cocobetaine, sodium cocoamphoacetate, (dimethicone•vinyldimethicone•methicone) cross-polymer, sodium hydroxide, stearamide DEA, stearic acid, potassium stearate, glyceryl stearate, sucrose stearate, sodium stearoyl methyl taurate, sodium stearoyl glutamate, sorbitan sesquioleate, (C12-14) pareth-12, (C12-15) pareth-2-phosphate, benzalconium chloride, polysorbate 80, polyquaternium-39, coconut oil fat, sodium cocoate, lauryl betaine, sodium lauryl sulfate, lauryltrimonium chloride, sodium laureth-12 sulfate, lecithin, etc.

A stabilizing component refers to a component that maintains quality at a fixed level, and examples include BHA, EDTA, PEG-2M, acrylic-based emulsifier/thickener, isostearic acid, karaya gum, carrageenan, carbomer, carnauba wax, agar, xanthan gum, candelilla wax, beef tallow, gluconolactone, crystalline cellulose, synthetic stevensite, cholesterol, cyclodextrin, cellulose gum, cetanol, ceresin, soy sterol, paraffin, pectin, bentonite, polyvinyl alcohol, polyethylene, microcrystalline wax, beeswax, methyl cellulose, sumac wax, pentaerythrityl rosinate, locust bean gum, etc.

An antimicrobial component refers to a component for preventing change of properties of the water-soluble electrospun sheet due to microorganisms, and examples include quaternium-73, calamus root extract, zinc pyrithione, tea tree oil, garlic extract, methyl paraoxybenzoate, phenoxyethanol, eucalyptus extract, resorcin, rosemary extract, etc.

A humectant component refers to a component that maintains the water content of skin epidermis and prevents evaporation of water from a keratinous layer, and examples include 1,3-butylene glycol, sodium DNA, propylene glycol, sodium di-pyrrolidonecarboxylate, sodium RNA, angelica keiskei extract, asparaginic acid, sweet tea extract, arginine, alanine, althaea extract, aloe vera extract-2, aloe ferox extract, aloe arborescens extract-2, oyster extract, persimmon leaf extract, hydrolyzed keratin, hydrolyzed conchiolin, hydrolyzed collagen, hydrolyzed albumen, hydrolyzed eggshell membrane, hydrolyzed silk, hydrolyzed soy protein, brown algae extract, Chinese quince extract, xylitol, raspberry extract, chitosan, cucumber extract, guava leaf extract, quince seed gum extract, glycine, glucose, glycerin, clematis extract, grapefruit extract, burdock root extract, rice fermentation extract, sodium chondroitin sulfate, fish collagen, hawthorn extract, rehmanniae radix extract, cystine, diglycerin, cysteine, horsetail extract, serine, malva sylvestris extract, sorbitol, soy fermentation extract, soy protein, tomato extract, trehalose, sodium lactate, urea, rosa canina extract, malt extract, honey, sodium hyaluronate, poria cocos extract, loofah extract, betaine, maltose, maltitol, mannitol, lily extract, lactoferrin, lysine, apple extract, astragalus extract, royal jelly, etc., but are not restricted thereto.

A skin-whitening component refers to a component that suppresses production of melanin pigments, and examples include t-AMCHA, ascorbic acid, arbutin, acerola extract, rosa multiflora extract, ellagic acid, chamomile extract, pyracantha fortuneana extract, kiwi extract, glutathione, ascorbyl tetrahexyldecanoate, tocotrienol, ferulic acid, raspberry ketone, rucinol, etc., but are not restricted thereto. An anti-ultraviolet component refers to a component having a function of protecting skin against ultraviolet rays and includes ultraviolet absorbing components and ultraviolet scattering components. Specific examples include t-butyl methoxydibenzoylmethane, oxybenzone-1,2,4-dihydroxybenzophenone, titanium oxide, cerium oxide, etc., but are not restricted thereto.

An astringent component refers to a component that provides a skin tightening sensation and suppresses sebum secretion, and examples include nettle leaf extract, eleutherococcus extract, aluminum chloride, sodium chloride, cork tree bark extract, sea salt, citric acid, coffee seed extract, succinic acid, betula alba extract, tartaric acid, peppermint extract, thyme extract, tea leaf extract, witch hazel extract, isodonis japonicus extract, coltsfoot extract, grape leaf extract, hop extract, horse chestnut extract, balm mint extract, etc., but are not restricted thereto.

A keratin-softening component refers to a component that softens a thickened and hardened keratinous layer, and examples include sulfur, glycolic acid, salicylic acid, lactic acid, papain, sodium sulfate, etc., but are not restricted thereto.

An anti-inflammatory component refers to a component that suppresses inflammation and prevents acne, skin roughness, etc., and examples include allantoin, arnica flower extract, coptis japonica root extract, scutellaria root extract, lamium album extract, typha angustifolia spike extract, calamine, chamomile extract, licorice extract, artemisia capillaris extract, gardenia florida extract, guaiazulene, bambuseae sasa extract, stearyl glycyrrhetinate, disodiumglycyrrhetinate gentiana extract, comfrey extract, black tea extract, tocopherol acetate, methyl salicylate, zinc oxide, perilla extract, lithospermum root extract, linden extract, peony root extract, meadowsweet extract, honeysuckle extract, ivy extract, sage extract, elderflower extract, yarrow extract, swertia japonica extract, mulberry root extract, calendula extract, loquat leaf extract, pyridoxine hydrochloride salt, peach leaf extract, centaurea cyanus flower extract, saxifraga sarmentosa extract, mugwort extract, lettuce extract, anthemis nobilis flower extract, sanguisorba extract, etc., but are not restricted thereto.

An emollient component is a substance that is refined from petrolatum or petroleum and refers to a component that protects the skin and prevents evaporation of water. Examples include almond oil, avocado oil, olive oil, orange roughy oil, oleic acid, carrot extract, cacao fat, sesame oil, safflower oil, camellia sinensis oil, dihydrocholesterol, squalane, cholesteryl stearate, ceramide 2, N-stearoyl-phytosphingosine (ceramide 3), evening primrose oil, sunflower oil, castor oil, grape seed oil, phytosphingosine, jojoba oil, macadamia nut oil, mink oil, mineral oil, meadowfoam oil, eucalyptus oil, lanolin, linoleic acid, rosehip oil, petrolatum, etc., but are not restricted thereto.

A coloring component refers particularly to that which is usable in cosmetics and is largely classified into organic synthetic pigments (tar pigments), natural pigments, and inorganic pigments. Examples include kaolin, carbon black, caramel, carmine, argentine, gold, ultramarine, titanium oxide, iron oxide (colcothar), iron oxide (yellow iron oxide), iron oxide (black iron oxide), talc, boron nitride, paprika pigment, henna, mica-titanium oxide, mica, laccaic acid, etc., but are not restricted thereto.

A cosmetic sheet refers to a sheet that is applicable to cosmetic facial masks, face washes, cosmetic toners, beauty serums, emulsions, creams, and other basic cosmetics aimed at conditioning skin quality itself, and to makeup cosmetics, such as foundations, eye pencils (eye blacks), eye shadows, eyeliners, lipsticks, glosses, blushes (cheek colors), powders, manicures, etc. Such cosmetics are provided as products prepared in the form of cosmetic facial masks to be adhered to the epidermis, or solids, creams, gels, liquids, etc.

The water-soluble electrospun sheet according to the present invention dissolves readily in water and thus enables such usage methods as (1) dissolving in water at a location of adhesion, as well as (2) dissolving in advance at another location (for example, on a palm or in a bottle, etc.) and thereafter applying spreadingly onto a facial surface, hand, foot, belly, breast, or other predetermined location.

A cosmetic facial mask refers to a type of cosmetic that is adhered onto the epidermis for a fixed purpose such as skin-whitening, moisturizing, skincare, aging care, etc. With a conventional cosmetic facial mask, a nonwoven fabric or other non-dissolving member is made to contain predetermined components, and thus in its usage, the facial mask is removed from the epidermis a while after adhesion. In this process, most of the effective components remain in the non-dissolving member and it is thus difficult to make effective use of the components. Also, although water-soluble cosmetic facial masks are now sold commercially (for example, Aura Skin (http://www.j-fc.co.jp/), a strong sticky sensation remains after dissolving on the epidermis and there is room for improvement of usage properties. Meanwhile, the cosmetic facial mask according to the present invention dissolves readily in water, and because it is thus dissolved rapidly after adhesion onto the epidermis by the water content in the epidermis (or by application of a small amount of water onto the epidermis by an atomizer after adhesion or by adhering onto the epidermis that has been put in a state of containing water in advance), there is no need to remove the facial mask. Also, the usability is improved by using, as a base material, a collagen peptide or other component compatible to the skin.

A cosmetic toner refers to a liquid cosmetic that moisturizes, conditions, or smoothens the skin and is also referred to as a lotion, toner, tonic, etc. In exceptional cases, a cosmetic toner is added to a foundation or a powder and used for the purpose of adjusting viscosity or improving ease of application. Types of cosmetic toners include general cosmetic toners (soft toners: used mainly after face washing to moisturize and prevent skin roughening), alkali toners (although most cosmetic toners are weakly acidic or neutral, there are some that are alkaline; Bälz water (glycerin and potash solution) is a representative example), astringent toners (astringent: an acidic lotion that temporarily suppresses the loss of sebum and conditions the texture by actions of an astringent agent); wipe-off toners (fresheners, removal toners: for removal of light cleansing cream, cold cream, or cleansing cream; used by absorbing into cotton, etc., and then wiping the skin for the purpose of cleansing the skin (face washing)), pre-toners (toners used before cosmetic toners), lotions (English translation for cosmetic toners and refers to colognes, hair tonics, and other alcohol-based liquid cosmetics in general), aftershave lotions (used after shaving to prevent skin roughening, razor rash, burning, etc.), carmine lotions (calamine, calamine lotions, type of astringent toner), body lotions (toners used on body parts besides the face), etc. Conventional toners are carried in a container when going out and are inconvenient in terms of portability due to being bulky, not being able to bring onto an airplane, etc. With the cosmetic toner sheet according to the present invention, just a necessary amount can be carried conveniently in sheet form and can be used immediately on the spot by dissolving in water and is thus not bulky and can be brought onto an airplane, etc. Also, a necessary amount can be prepared whenever necessary, and thus a cosmetic toner of a concentration of choice can be prepared and can also be used in a fresh state.

A beauty serum refers to a liquid in which a humectant component, skin-whitening component, or other beauty component is formulated in concentrated form, and in many cases a beauty serum is used after conditioning the skin with a cosmetic toner, etc., and before using an oil-containing basic cosmetic to increase absorption of the beauty component into the skin. The beauty serum according to the present invention provides the same effects as the cosmetic toner described above.

In addition to that which is used by a doctor or dentist in performing a medical treatment, a medical sheet also includes that used in a minor procedure (for example, a procedure for treating a minor abrasion, cut, etc.,) in a household; etc. The medical sheet according to the present invention dissolves readily in water and thus enables such usage methods as (1) dissolving in water at a location of adhesion, as well as (2) dissolving in advance at another location (for example, on a fingertip, etc.,) and thereafter applying spreadingly onto a facial surface, hand, foot, belly, breast, or other predetermined location. The medical sheet can specifically be applied to either or both of a sheet provided with an antimicrobial substance and having an antimicrobial (antibacterial) action and a wound treatment sheet provided with an anti-inflammatory substance and having an anti-inflammatory action, etc.

Although with many conventional antimicrobial sterilizers (for example, Makiron), the drug solution is applied to an affected portion upon soaking into a gauze or absorbent cotton, this procedure is accompanied by pain in many cases because the wound is contacted directly. Although there are also spray type antimicrobials that are sprayed onto the affected portion, such spraying also is accompanied by pain in many cases because the antimicrobial is sprayed strongly onto the wound. Further in many cases, a more-than-necessary amount is sprayed, causing dripping of liquid.

The medical sheet according to the present invention can be cut according to a size of a wound and just the necessary amount can be adhered. By adhering in a wet state after washing of the wound, the sheet dissolves instantaneously to enable the medical components to be applied to the affected portion without sensation of pain.

The water-soluble electrospun sheet refers to a sheet made of the water-soluble base material that has been formed into fibers. Here, a fiber refers to a filament with a single-yarn diameter of 10 nm to several 10 μm. By the electrospinning method, a nonwoven fabric is obtained as a two-dimensional aggregate, that is, as a sheet.

To prepare the water-soluble electrospun sheet according to the present invention, first the water-soluble base material is dissolved in a suitable solvent and the electrospinning method is thereafter carried out using this solution. In this process, a suitable functional component can be mixed in addition to the water-soluble base material. A mixing proportion of the functional component is not restricted in particular and can be set suitably according to properties of the water-soluble base material and the functional component. Also, as the solvent, an inorganic solvent, such as water, etc., an organic solvent, such as alcohol, acetone, etc. (including protic polar solvents and aprotic polar solvents), etc., can be used. Here, in a case where a cosmetic or other product that is used upon adhering directly onto the epidermis is to be manufactured, water or ethanol is preferably used in consideration of safety. The electrospinning method is influenced by such factors as concentration of the base material, type of solvent, needle gauge, ejection distance, rotation speed, voltage, ejection rate, etc. Actual manufacture of a sheet can be carried out by suitably combining the above factors.

The water-soluble electrospun sheet that is thus manufactured dissolves readily in water and can be used as various materials, such as cosmetic sheets (including cosmetic facial masks, cosmetic toners, and beauty serums), medical sheets, etc. Also, by making another functional component (for example, a humectant component, skin-whitening component, anti-ultraviolet component, astringent component, keratin-softening component, anti-inflammatory component, coloring component, etc.,) be contained in addition to the base material, a specific function can be exhibited at a site onto which the sheet is adhered or a site at which the component is rubbed in after dissolution of the sheet.

Although the present invention shall now be described in detail byway of examples and test examples, the present invention is not restricted by these examples and test examples.

### Example 1

### Preparation of Collagen Peptide Nanofibers

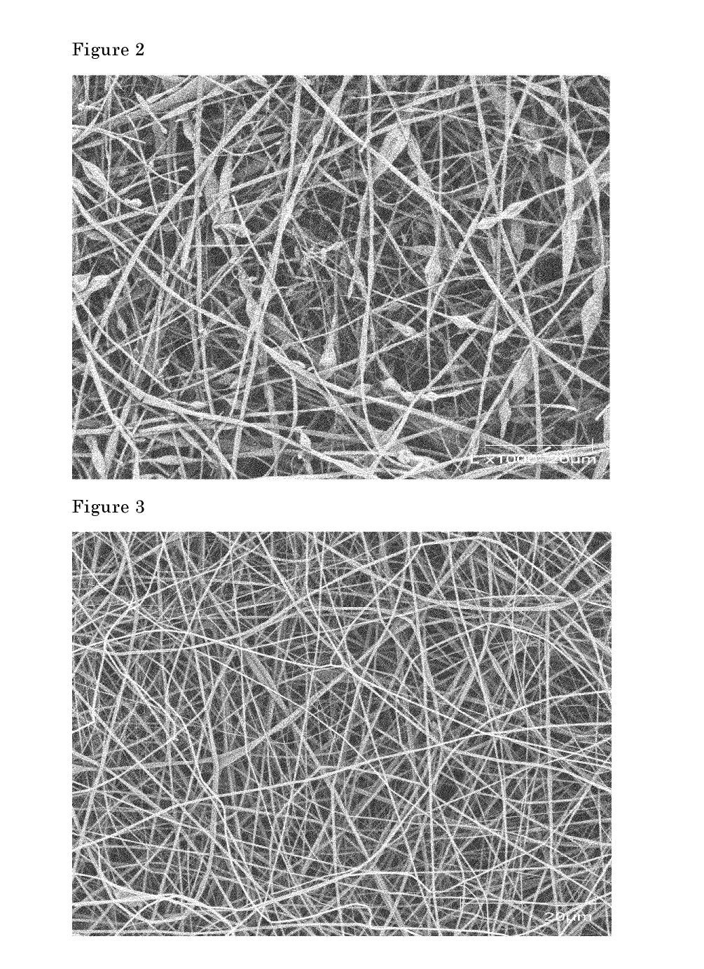

4.5 g of pig skin collagen peptide (collagen peptide PCH (average molecular weight: 5,000) made by Unitec Foods Co., Ltd.) and 5.5 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. A collagen peptide solution with a collagen peptide concentration of 45 mass % was thus obtained. The collagen peptide solution was loaded into a syringe (made by Terumo Corp.), a 23 G needle (made by Hoshiseido Medical Instrumentation Co., Ltd.) was attached to a tip of the syringe, and air bubbles inside the syringe were removed completely. The syringe was set on a syringe pump of an electrospinning apparatus (made by Imoto Machinery Co., Ltd.), and spinning was carried out under the spinning conditions described below. As a result, a collagen peptide nanofiber sheet with an average fiber diameter of approximately 0.20 to 1.5 μm was obtained. An electron micrograph is shown in FIG. 2.

Spinning conditions

| | |
|---|---|
| Voltage | 20 kV |
| Ejection rate | 2 ml/hr |
| Ejection distance | 10 cm |
| Roller rotation speed | 80 rpm |
| Temperature inside apparatus | 20 to 25° C. |
| Humidity inside apparatus | no more than 50% |

### Example 2

### Preparation of Gelatin Nanofibers

1.5 g of gelatin (Neosoft GE-388 made by Taiyo Kagaku Co., Ltd.) and 8.5 g of ion-exchanged water were placed and sealed in a sample vial, and complete dissolution was achieved by performing vibration stirring while warming. A gelatin solution with a gelatin concentration of 15 mass % was thus obtained. The gelatin solution was loaded into a syringe (made by Terumo Corp.), an 18 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the spinning conditions as in Example 1. As a result, a gelatin nanofiber sheet with an average fiber outer diameter of approximately 0.15 to 2.0 μm was obtained. An electron micrograph is shown in FIG. 3.

### Example 3

### Preparation of HPC Nanofibers

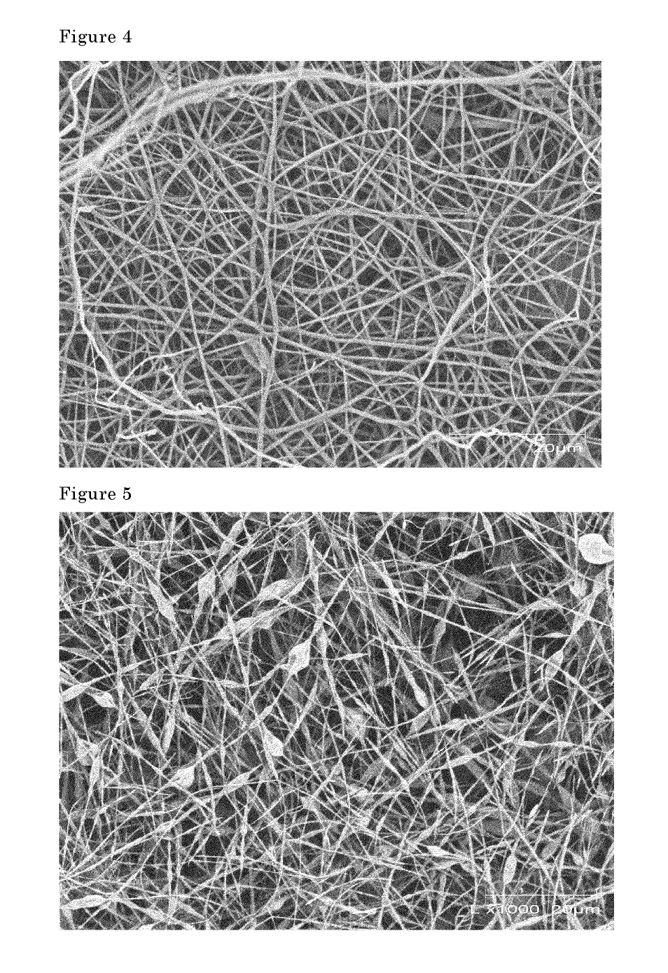

0.3 g of HPC (hydroxypropyl cellulose (H) made by Nippon Soda Co., Ltd.) and 9.7 g of ethanol (ethanol (no less than 99%, first grade, fermented) made by Japan Alcohol Trading Co., Ltd.) were placed and sealed in a sample vial, and complete dissolution was achieved by performing vibration stirring while warming. An HPC solution with an HPC concentration of 3 mass % was thus obtained. The HPC solution was loaded into a syringe (made by Terumo Corp.), a 25 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the spinning conditions as in Example 1. As a result, an HPC nanofiber sheet with an average fiber outer diameter of approximately 0.10 to 1.0 μm was obtained. An electron micrograph is shown in FIG. 4.

### Example 4

### Preparation of Sodium Hyaluronate Nanofibers

0.1 g of sodium hyaluronate (sodium hyaluronate made by Wako Pure Chemical Industries, Ltd.) and 9.9 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. A sodium hyaluronate solution with a sodium hyaluronate concentration of 1 mass % was thus obtained. The sodium hyaluronate solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the spinning conditions described below. As a result, a sodium hyaluronate nanofiber sheet with an average fiber outer diameter of approximately 0.1 to 2.0 μm was obtained. An electron micrograph is shown in FIG. 5.

Spinning conditions

| | |
|---|---|
| Voltage | 25 kV |
| Ejection rate | 2 ml/hr |
| Ejection distance | 10 cm |
| Roller rotation speed | 80 rpm |
| Temperature inside apparatus | 20 to 25° C. |
| Humidity inside apparatus | no more than 50% |

### Example 5

### Preparation of Quince Seed Gum Nanofibers

0.3 g of quince seed gum (quince seed gum made by Taiyo Kagaku Co. , Ltd.) and 9.7 g of ethanol (ethanol (no less than 99%, first grade, fermented) made by Japan Alcohol Trading Co., Ltd.) were placed and sealed in a sample vial, and complete dissolution was achieved by performing vibration stirring while warming. A quince seed gum solution with a quince seed gum concentration of 3 mass % was thus obtained. The quince seed gum solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the spinning conditions described below. As a result, a quince seed gum nanofiber sheet with an average fiber outer diameter of approximately 0.2 to 2.2 μm was obtained.

Spinning conditions

| | |
|---|---|
| Voltage | 20 kV |
| Ejection rate | 1 ml/hr |
| Ejection distance | 10 cm |
| Roller rotation speed | 80 rpm |
| Temperature inside apparatus | 20 to 25° C. |
| Humidity inside apparatus | no more than 50% |

### Example 6

### Preparation of PVA Nanofibers

2.5 g of PVA (polyvinyl alcohol 3,500 made by Wako Pure Chemical Industries, Ltd.) and 7.5 g of ion-exchanged water were placed and sealed in a sample vial, and complete dissolution was achieved by performing vibration stirring while warming. A PVA solution with a PVA concentration of 25 mass % was thus obtained. The PVA solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the spinning conditions described below. As a result, a PVA nanofiber sheet with an average fiber outer diameter of approximately 0.2 to 2.5 μm was obtained.

Spinning conditions

| | |
|---|---|
| Voltage | 15 kV |
| Ejection rate | 1 ml/hr |
| Ejection distance | 15 cm |
| Roller rotation speed | 80 rpm |
| Temperature inside apparatus | 20 to 25° C. |
| Humidity inside apparatus | no more than 50% |

### Example 7

### Preparation of Sodium Polyacrylate Nanofibers

0.1 g of sodium polyacrylate (Viscomate made by Showa Denko K. K.) and 7.5 g of ion-exchanged water were placed and sealed in a sample vial, and complete dissolution was achieved by performing vibration stirring while warming. A sodium polyacrylate solution with a sodium polyacrylate concentration of 1 mass % was thus obtained. The sodium polyacrylate solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the spinning conditions described below. As a result, a sodium polyacrylate nanofiber sheet with an average fiber outer diameter of approximately 0.1 to 1.5 μm was obtained.

Spinning conditions

| | |
|---|---|
| Voltage | 17.5 kV |
| Ejection rate | 2 ml/hr |
| Ejection distance | 10 cm |
| Roller rotation speed | 80 rpm |
| Temperature inside apparatus | 20 to 25° C. |
| Humidity inside apparatus | no more than 50% |

### Example 8

### Preparation of Silk Fibroin Nanofibers

5.0 g of silk fibroin (silk fibroin made by Silk Kogei K. K.) and 5.0 g of 30 w/w % ethanol (ethanol (no less than 99%, first grade, fermented) made by Japan Alcohol Trading Co., Ltd.; ethanol:water mass ratio=30:70) were placed and sealed in a sample vial, and complete dissolution was achieved by performing vibration stirring while warming. A silk fibroin solution with a silk fibroin concentration of 50 mass % was thus obtained. The silk fibroin solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the spinning conditions described below. As a result, a silk fibroin nanofiber sheet with an average fiber outer diameter of approximately 0.3 to 2.8 μm was obtained.

Spinning conditions

| | |
|---|---|
| Voltage | 20 kV |
| Ejection rate | 3 ml/hr |
| Ejection distance | 10 cm |
| Roller rotation speed | 80 rpm |
| Temperature inside apparatus | 20 to 25° C. |
| Humidity inside apparatus | no more than 50% |

### Example 9

### Preparation of Water-Soluble Chitosan Nanofibers

2.5 g of water-soluble chitosan (chitosan, water-soluble, made by Wako Pure Chemical Industries, Ltd.) and 7.5 g of ion-exchanged water were placed and sealed in a sample vial, and complete dissolution was achieved by performing vibration stirring while warming. A chitosan solution with a chitosan concentration of 25 mass % was thus obtained. The chitosan solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the spinning conditions described below. As a result, a chitosan nanofiber sheet with an average fiber outer diameter of approximately 0.2 to 2.5 μm was obtained.

### Comparative Example 1

As Comparative Example 1, a commercially-sold soluble film (Aura Skin Film face mask, made by Tsukioka Co., Ltd.) was used.

### Comparative Example 2

As Comparative Example 2, a commercially-sold edible film (Extra Mint breath-care film, made by Kobayashi Pharmaceutical Co., Ltd.) was used.

### Test Example 1

### Solubility Test

Each of the sheets of Examples 1 to 9 and the sheets of Comparative Examples 1 and 2 were cut into a size of 3 cm×3 cm and immersed in a 200 ml beaker containing 100 ml of ion-exchanged water of 25 G, and the time for complete dissolution was measured. Rating was performed according to the following five stages as evaluation standards. That is, the five stages are: 5: dissolved within 1 second; 4: dissolved within 2 seconds to 10 seconds; 3: dissolved within 11 seconds to 30 seconds; 2: dissolved within 31 seconds to 60 seconds; and 1: not less than 61 seconds required for dissolution. The results are shown in Table 1.

TABLE 1

| | | | | | | | | | | Comparative | |
|---|---|---|---|---|---|---|---|---|---|---|---|
| | Example | | | | | | | | | Example | |
| | 1 | 2 | 3 | 4 | 5 | 6 | 7 | 8 | 9 | 1 | 2 |
| Solubility | 5 | 4 | 4 | 5 | 4 | 4 | 5 | 4 | 5 | 3 | 2 |

The sheets of all of Examples 1 to 9 dissolved completely and rapidly after contacting water. On the other hand, with the sheets of Comparative Examples 1 and 2, several dozen seconds were required for complete dissolution. The sheets of all of Examples 1 to 9 are formed to sheets of nanometer-order fibers and are thus large in contact area with water molecules, and these sheets were found to be excellent in solubility in comparison to Comparative Examples 1 and 2.

### Example 10

### Preparation of Collagen Peptide/Theanine Nanofibers

4.5 g of pig skin collagen peptide (collagen peptide PCH made by Unitec Foods Co., Ltd.) and 5.5 g of 50 w/w % ethanol (ethanol (no less than 99%, first grade, fermented) made by Japan Alcohol Trading Co., Ltd.; ethanol:water mass ratio=50:50) were placed and sealed in a sample vial 3, and complete dissolution was achieved by performing vibration stirring while warming. After dissolution of the collagen peptide, 0.045 g of theanine (Suntheanine made by Taiyo Kagaku Co., Ltd.) were added, and complete dissolution was achieved by further performing vibration stirring under room temperature. A collagen peptide/theanine solution with a collagen peptide concentration of 45 mass % and a theanine concentration of 0.45 mass % was thus obtained. The collagen peptide/theanine solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1. As a result, a collagen peptide/theanine nanofiber sheet with an average fiber outer diameter of approximately 0.1 to 2.1m was obtained. A nanofiber sheet was thus obtained with which, theoretically, 1% of theanine is blended with respect to the mass of the collagen peptide.

### Example 11

### Preparation of Collagen Peptide/Hyaluronic Acid Nanofibers

9.0 g of pig skin collagen peptide (collagen peptide PCH made by Unitec Foods Co., Ltd.) and 11 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. After dissolution of the collagen peptide, 0.0009 g of hyaluronic acid (sodium hyaluronate made by Wako Pure Chemical Industries, Ltd.) were added, and complete dissolution was achieved by further performing vibration stirring under room temperature. A collagen peptide/hyaluronic acid solution with a collagen peptide concentration of 45 mass % and a hyaluronic acid concentration of 0.0045 mass % was thus obtained. The collagen peptide/hyaluronic acid solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a collagen peptide/hyaluronic acid nanofiber sheet with an average fiber outer diameter of approximately 0.10 to 2.0 μm was obtained. A nanofiber sheet was thus obtained with which, theoretically, 0.01% of hyaluronic acid is blended with respect to the mass of the collagen peptide.

### Comparative Example 3

### Preparation of Collagen Peptide/Zein/Theanine Nanofibers

2.25 g of pig skin collagen peptide (collagen peptide PCH made by Unitec Foods Co., Ltd.), 2.25 g of zein (Kobayashi Zein DP made by Kobayashi Pharmaceutical Co., Ltd.), and 5.5 g of 50 w/w % ethanol (ethanol (no less than 99%, first grade, fermented) made by Japan Alcohol Trading Co., Ltd.; ethanol:water mass ratio=50:50) were placed and sealed in a sample vial 3, and complete dissolution was achieved by performing vibration stirring while warming. After dissolution of the collagen peptide and zein, 0.045 g of theanine (Suntheanine made by Taiyo Kagaku Co., Ltd.) were added, and complete dissolution was achieved by further performing vibration stirring under room temperature. A collagen peptide/zein/theanine solution with a collagen peptide concentration of 22.5 mass %, a zein concentration of 22.5 mass %, and a theanine concentration of 0.45 mass % was thus obtained. The collagen peptide/zein/theanine solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a collagen peptide/theanine nanofiber sheet with an average fiber outer diameter of approximately 0.10 to 2.0 μm was obtained. A nanofiber sheet was thus obtained with which, theoretically, 1% of theanine is blended with respect to the total mass of the collagen peptide and zein.

### Comparative Example 4

### Preparation of Zein/Theanine Nanofibers

4.5 g of zein (Kobayashi Zein DP made by Kobayashi Pharmaceutical Co., Ltd.) and 5.5 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. After dissolution of the collagen, 0.045 g of theanine (Suntheanine made by Taiyo Kagaku Co., Ltd.) were added, and complete dissolution was achieved by further performing vibration stirring under room temperature. A collagen peptide/theanine solution with a collagen peptide concentration of 45 mass % and a theanine concentration of 0.045 mass % was thus obtained. The collagen peptide/theanine solution was loaded into a syringe (made by Terumo Corp.), a 21 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a collagen peptide/theanine nanofiber sheet with an average fiber outer diameter of approximately 0.10 to 1.5 μm was obtained. A nanofiber sheet was thus obtained with which, theoretically, 1% of theanine is blended with respect to the mass of zein.

### Comparative Example 5

### Preparation of Collagen Peptide/Zein/Hyaluronic Acid Nanofibers

4.5 g of pig skin collagen peptide (collagen peptide PCH made by Unitec Foods Co., Ltd.), 4.5 g of zein (Kobayashi Zein DP made by Kobayashi Pharmaceutical Co., Ltd.), and 11 g of 50 w/w % ethanol (ethanol (no less than 99%, first grade, fermented) made by Japan Alcohol Trading Co., Ltd. ; ethanol:water mass ratio=50:50) were placed and sealed in a sample vial, and complete dissolution was achieved by performing vibration stirring while warming. After dissolution of the collagen peptide and zein, 0.0009 g of hyaluronic acid (sodium hyaluronate made by Wako Pure Chemical Industries, Ltd.) were added, and complete dissolution was achieved by further performing vibration stirring under room temperature. A collagen peptide/zein/hyaluronic acid solution with a collagen peptide concentration of 22.5 mass %, a zein concentration of 22.5 mass %, and a hyaluronic acid concentration of 0.0045 mass % was thus obtained. The collagen peptide/zein/hyaluronic acid solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a collagen peptide/zein/hyaluronic acid nanofiber sheet with an average fiber outer diameter of approximately 0.10 to 2.0 μm was obtained. A nanofiber sheet was thus obtained with which, theoretically, 0.01% of hyaluronic acid is blended with respect to the total mass of the collagen peptide and zein.

### Comparative Example 6

### Preparation of Zein/Hyaluronic Acid Nanofibers

9.0 g of zein (Kobayashi Zein DP made by Kobayashi Pharmaceutical Co., Ltd.) and 11 g of 50 w/w % ethanol (ethanol (no less than 99%, first grade, fermented) made by Japan Alcohol Trading Co., Ltd.; ethanol:water mass ratio=50:50) were placed and sealed in a sample vial 3, and complete dissolution was achieved by performing vibration stirring while warming. After dissolution of the collagen, 0.0009 g of hyaluronic acid (sodium hyaluronate made by Wako Pure Chemical Industries, Ltd.) were added, and complete dissolution was achieved by further performing vibration stirring under room temperature. A collagen peptide/theanine solution with a collagen peptide concentration of 45 mass % and a hyaluronic acid concentration of 0.0045 mass % was thus obtained. The zein/hyaluronic acid solution was loaded into a syringe (made by Terumo Corp.), a 21 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a zein/hyaluronic acid nanofiber sheet with an average fiber outer diameter of approximately 0.10 to 1.8 μm was obtained. A nanofiber sheet was thus obtained with which, theoretically, 0.01% of hyaluronic acid is blended with respect to the mass of zein.

### Test Example 2

### Evaluation by Usage Test

Each of the sheets of Examples 9 and 10 and the sheets of Comparative Examples 3 to 6 were cut into a size of 5 cm×5 cm and adhered to cheeks of ten panelists, approximately 1 mL of ion-exchanged water was sprayed on uniformly using a sprayer, and dissolution states of the sheets were observed.

As a result, the sheets dissolved immediately after spraying and pieces of the sheets did not remain on the skin. Each panelist performed the operation of dissolving on the skin by this method once a day before going to bed, and after continuing the operation for one week, expressed various evaluations of the skin state according to the five stages of 1 to 5 described below, and in a final stage, average values of points of all panelists were determined as the evaluation results. The evaluation items are the six items of speed of dissolution, ease of use, feeling of exhilaration upon use, smoothness of skin, moisturizing effect, and skin irritation. Comparative Example 1 and Comparative Example 2 were used as comparison controls.

Evaluation Item (1) was the speed of dissolution. The evaluation was made according to the five stages of: 5: dissolved immediately without touching after spraying on with the sprayer; 4: dissolved after several seconds without touching after spraying on with the sprayer; 3: dissolved after several seconds upon spreading with the hand after spraying on with the sprayer; 2: dissolved after several dozen seconds upon spreading with the hand after spraying on with the sprayer; and 1: non-dissolved residue remained even after spreading with the hand after spraying on with the sprayer.

Evaluation Item (2) was the ease of use. The evaluation was made according to the five stages of: 5: extremely easy to use; 4: easy to use; 3: seems to be easy to use; 2: cannot say whether use is easy or difficult; and 1: difficult to use.

Evaluation Item (3) was the feeling of exhilaration upon use. The evaluation was made according to the five stages of: 5: extreme exhilaration was felt after use; 4: exhilaration was felt after use; 3: there seems to be exhilaration after use; 2: cannot say whether or not there is exhilaration after use; and 1: discomfort was felt after use.

Evaluation Item (4) was the smoothness of skin. The evaluation was made according to the five stages of: 5: the skin became significantly smooth after use; 4: the skin became smooth after use; 3: the skin seemed to become smooth after use; 2: cannot say whether or not the skin became smooth after use; and 1: the state of the skin worsened after use.

Evaluation Item (5) was the moisturizing effect. The evaluation was made according to the five stages of: 5: the skin became significantly smooth after use; 4: the skin became smooth after use; 3: the skin seemed to become smooth after use; 2: cannot say whether or not the skin became smooth after use; and 1: the state of the skin worsened after use.

Evaluation Item (6) was the skin irritation. The evaluation was made according to the five stages of: 5: no irritation whatsoever was felt after use; 4: hardly any irritation was felt after use; 3: slight irritation was felt after use; 2: irritation was felt after use; and 1: significant irritation was felt after use.

The results are shown in Table 2.

TABLE 2

| | | Example | | Comparative Example | | | |
|---|---|---|---|---|---|---|---|
| Evaluation Item | | 10 | 11 | 3 | 4 | 5 | 6 |
| (1) | Speed of dissolution | 5 | 5 | 1 | 1 | 1 | 1 |
| (2) | Ease of use | 5 | 5 | 3 | 2 | 3 | 2 |
| (3) | Feeling of exhilaration | 5 | 5 | 3 | 3 | 3 | 2 |
| | upon use | | | | | | |
| (4) | Smoothness of skin | 5 | 5 | 4 | 3 | 4 | 3 |
| (5) | Moisturizing effect | 5 | 5 | 3 | 3 | 3 | 3 |
| (6) | Skin irritation | 5 | 5 | 5 | 3 | 5 | 3 |

As described above, in comparison to the sheets of Comparative Examples 3 to 6, the usability of each of the sheets of Examples 10 and 11 was extremely favorable in terms of ease of use and feeling of exhilaration. A lack of a need to peel and immediate dissolution are the significant characteristics of these examples. The sheets of the examples are not applied spreadingly onto the skin using a finger, etc., and suffice to be simply adhered to the location of use, and thus there is no attachment to a finger, etc. Also, the examples are extremely good as facial masks and other cosmetic forms in that the effective components can be supplied adequately, uniformly, and efficiently along with a small amount of water and be made to act effectively on the skin.

Besides the examples described above, various nanofiber sheets having cosmetic materials and quasi-drug materials blended therein were prepared, and details thereof are described in the following examples.

### Example 12

### Preparation of Collagen Peptide/Vitamin C Nanofibers

4.5 g of pig skin collagen peptide (collagen peptide PCH made by Unitec Foods Co., Ltd.) and 5.5 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. After dissolution of the collagen peptide, 0.135 g of vitamin C (sodium ascorbate made by Tanabe Pharma Corp.) were added, and complete dissolution was achieved by further performing vibration stirring under room temperature. A collagen peptide/vitamin C solution with a collagen peptide concentration of 45 mass % and a vitamin C concentration of 1.35 mass % was thus obtained. The collagen peptide/vitamin C solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a collagen peptide/vitamin C nanofiber sheet with an average fiber outer diameter of approximately 0.10 to 2.0 μm was obtained. A nanofiber sheet was thus obtained with which, theoretically, 3% of vitamin C is blended with respect to the mass of the collagen peptide.

### Example 13

### Preparation of HPC/CoQ10 Nanofibers

0.3 g of HPC (hydroxypropyl cellulose (H) made by Nippon Soda Co., Ltd.) and 9.7 g of ethanol (ethanol (no less than 99%, first grade, fermented) made by Japan Alcohol Trading Co., Ltd.) were placed and sealed in a sample vial, and complete dissolution was achieved by performing vibration stirring while warming. After dissolution of the HPC, 0.00027 g of a CoQ10 formulation (SUN ACTIVE Q-10Y, with a CoQ10 concentration of 10 mass %; made by Taiyo Kagaku Co., Ltd.) were added, and complete dissolution was achieved by further performing vibration stirring under room temperature. An HPC/CoQ10 solution with an HPC concentration of 3 mass % and a CoQ10 concentration of 0.0027 mass % was thus obtained. The HPC/CoQ10 solution was loaded into a syringe (made by Terumo Corp.), an 18 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, an HPC/CoQ10 nanofiber sheet with an average fiber outer diameter of approximately 0.2 to 2 μm was obtained. A nanofiber sheet was thus obtained with which, theoretically, 0.03% of CoQ10 is blended with respect to the mass of HPC.

### Example 14

### Preparation of PVA/urea Nanofibers

2.5 g of PVA (polyvinyl alcohol 3,500 made by Wako Pure Chemical Industries, Ltd.) and 7.5 g of ion-exchanged water were placed and sealed in a sample vial, and complete dissolution was achieved by performing vibration stirring while warming. After dissolution of the PVA, 0.025 g of urea were added, and complete dissolution was achieved by further performing vibration stirring under room temperature. A PVA/urea solution with a PVA concentration of 25 mass % and a urea concentration of 0.25 mass % was thus obtained. The PVA/urea solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 6.

As a result, a PVA/urea nanofiber sheet with an average fiber outer diameter of approximately 0.1 to 2.5 μm was obtained. A nanofiber sheet was thus obtained with which, theoretically, 1% of urea is blended with respect to the mass of PVA.

### Example 15

### Preparation of PVA/Hydrolyzed Eggshell Membrane Nanofibers

2.5 g of PVA (polyvinyl alcohol 3,500 made by Wako Pure Chemical Industries, Ltd.) and 7.5 g of ion-exchanged water were placed and sealed in a sample vial, and complete dissolution was achieved by performing vibration stirring while warming. After dissolution of the PVA, 0.0025 g of hydrolyzed eggshell membrane were added, and complete dissolution was achieved by further performing vibration stirring under room temperature. A PVA/hydrolyzed eggshell membrane solution with a PVA concentration of 25 mass % and a hydrolyzed eggshell membrane concentration of 0.025 mass % was thus obtained. The PVA/hydrolyzed eggshell membrane solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 6.

As a result, a PVA/hydrolyzed eggshell membrane nanofiber sheet with an average fiber outer diameter of approximately 0.1 to 2.5 μm was obtained. A nanofiber sheet was thus obtained with which, theoretically, 0.1% of hydrolyzed eggshell membrane is blended with respect to the mass of PVA.

### Example 16

### Preparation of HPC/Sodium Chondroitin Sulfate Nanofibers

0.3 g of HPC (hydroxypropyl cellulose (H) made by Nippon Soda Co., Ltd.) and 9.7 g of ethanol (ethanol (no less than 99%, first grade, fermented) made by Japan Alcohol Trading Co., Ltd.) were placed and sealed in a sample vial, and complete dissolution was achieved by performing vibration stirring while warming. After dissolution of the HPC, 0.003 g of sodium chondroitin sulfate (sodium chondroitin sulfate C made by Wako Pure Chemical Industries, Ltd.) were added, and complete dissolution was achieved by further performing vibration stirring under room temperature. An HPC/sodium chondroitin sulfate solution with an HPC concentration of 3 mass % and a sodium chondroitin sulfate concentration of 0.03 mass % was thus obtained. The HPC/sodium chondroitin sulfate solution was loaded into a syringe (made by Terumo Corp.), an 18 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 6.

As a result, an HPC/sodium chondroitin sulfate nanofiber sheet with an average fiber outer diameter of approximately 0.15 to 2.0 μm was obtained. A nanofiber sheet was thus obtained with which, theoretically, 1% of sodium chondroitin sulfate is blended with respect to the mass of HPC.

### Example 17

### Preparation of Collagen Peptide/Glycol Salicylate Nanofibers

4.5 g of pig skin collagen peptide (collagen peptide PCH made by Unitec Foods Co., Ltd.) and 5.5 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. After dissolution of the collagen peptide, 0.045 g of glycol salicylate (Saliment made by ABI Corporation) were added, and complete dissolution was achieved by further performing vibration stirring under room temperature. A collagen peptide/glycol salicylate solution with a collagen peptide concentration of 45 mass % and a glycol salicylate concentration of 10 mass % was thus obtained. The collagen peptide/glycol salicylate solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a collagen peptide/glycol salicylate nanofiber sheet with an average fiber outer diameter of approximately 0.2 to 2.2 μm was obtained. A nanofiber sheet was thus obtained with which, theoretically, 1% of glycol salicylate is blended with respect to the mass of HPC.

### Example 18

### Preparation of Collagen Peptide/Diphenhydramine Hydrochloride Nanofibers

4.5 g of pig skin collagen peptide (collagen peptide PCH made by Unitec Foods Co., Ltd.) and 5.5 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. After dissolution of the collagen peptide, 0.009 g of diphenhydramine hydrochloride (diphenhydramine hydrochloride salt made by Tokyo Chemical Industry Co. Ltd.) were added, and complete dissolution was achieved by further performing vibration stirring under room temperature. A collagen peptide/diphenhydramine hydrochloride solution with a collagen peptide concentration of 45 mass % and a diphenhydramine hydrochloride concentration of 0.09 mass % was thus obtained. The collagen peptide/diphenhydramine hydrochloride solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a collagen peptide/diphenhydramine hydrochloride nanofiber sheet with an average fiber outer diameter of approximately 0.1 to 2.0 μm was obtained. A nanofiber sheet was thus obtained with which, theoretically, 0.2% of diphenhydramine hydrochloride is blended with respect to the mass of the collagen peptide.

### Example 19

### Preparation of HPC/Glycerin Nanofibers

0.25 g of HPC (hydroxypropyl cellulose (H) made by Nippon Soda Co., Ltd.) and 9.75 g of ethanol (ethanol (no less than 99%, first grade, fermented) made by Japan Alcohol Trading Co., Ltd.) were placed and sealed in a sample vial, and complete dissolution was achieved by performing vibration stirring while warming. After dissolution of the HPC, 0.25 g of glycerin (glycerin made by Wako Pure Chemical Industries, Ltd.) were added, and complete dissolution was achieved by further performing vibration stirring under room temperature. An HPC/glycerin solution with an HPC concentration of 2.5 mass % and a glycerin concentration of 2.5 mass % was thus obtained. The HPC/glycerin solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, an HPC/glycerin nanofiber sheet with an average fiber outer diameter of approximately 0.2 to 2.8 μm was obtained. A nanofiber sheet was thus obtained with which, theoretically, 100% of glycerin is blended with respect to the mass of HPC.

### Example 20

### Preparation of HPC/1,3-Butylene Glycol Nanofibers

0.25 g of HPC (hydroxypropyl cellulose (H) made by Nippon Soda Co., Ltd.) and 9.75 g of ethanol (ethanol (no less than 99%, first grade, fermented) made by Japan Alcohol Trading Co., Ltd.) were placed and sealed in a sample vial, and complete dissolution was achieved by performing vibration stirring while warming. After dissolution of the HPC, 0.25 g of 1,3-butylene glycol (1,3-butanediol made by Wako Pure Chemical Industries, Ltd.) were added, and complete dissolution was achieved by further performing vibration stirring under room temperature. An HPC/1,3-butylene glycol solution with an HPC concentration of 2.5 mass % and a 1,3-butylene glycol concentration of 2.5 mass % was thus obtained. The HPC/1,3-butylene glycol solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, an HPC/1,3-butylene glycol nanofiber sheet with an average fiber outer diameter of approximately 0.2 to 3.0 μm was obtained. A nanofiber sheet was thus obtained with which, theoretically, 100% of 1,3-butylene glycol is blended with respect to the mass of HPC.

### Example 21

### Preparation of Collagen Peptide/Arbutin Nanofibers

4.5 g of pig skin collagen peptide (collagen peptide PCH made by Unitec Foods Co., Ltd.) and 5.5 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. After dissolution of the collagen peptide, 0.045 g of arbutin (standard arbutin made by Wako Pure Chemical Industries, Ltd.) were added, and complete dissolution was achieved by further performing vibration stirring under room temperature. A collagen peptide/arbutin solution with a collagen peptide concentration of 45 mass % and an arbutin concentration of 0.45 mass % was thus obtained. The collagen peptide/arbutin solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a collagen peptide/arbutin nanofiber sheet with an average fiber outer diameter of approximately 0.10 to 2.0 μm was obtained. A nanofiber sheet was thus obtained with which, theoretically, 1% of arbutin is blended with respect to the mass of the collagen peptide.

### Example 22

### Preparation of HPC/Ellagic Acid Nanofibers

0.3 g of HPC (hydroxypropyl cellulose (H) made by Nippon Soda Co., Ltd.) and 9.7 g of ethanol (ethanol (no less than 99%, first grade, fermented) made by Japan Alcohol Trading Co., Ltd.) were placed and sealed in a sample vial, and complete dissolution was achieved by performing vibration stirring while warming. After dissolution of the HPC, 0.0009 g of titanium oxide (ellagic acid dihydrate made by Wako Pure Chemical Industries, Ltd.) were added, and complete dissolution was achieved by further performing vibration stirring under room temperature. An HPC/ellagic acid solution with an HPC concentration of 3 mass % and an ellagic acid concentration of 0.009 mass % was thus obtained. The HPC/ellagic acid solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, an HPC/ellagic acid nanofiber sheet with an average fiber outer diameter of approximately 0.2 to 2.0 μm was obtained. A nanofiber sheet was thus obtained with which, theoretically, 0.3% of ellagic acid is blended with respect to the mass of HPC.

### Example 23

### Preparation of HPC/2,4-Dihydroxybenzophenone Nanofibers

0.3 g of HPC (hydroxypropyl cellulose (H) made by Nippon Soda Co., Ltd.) and 9.7 g of ethanol (ethanol (no less than 99%, first grade, fermented) made by Japan Alcohol Trading Co., Ltd.) were placed and sealed in a sample vial, and complete dissolution was achieved by performing vibration stirring while warming. After dissolution of the HPC, 0.03 g of 2,4-dihydroxybenzophenone (2,4-dihydroxybenzophenone made by Wako Pure Chemical Industries, Ltd.) were added, and complete dissolution was achieved by further performing vibration stirring under room temperature. An HPC/2,4-dihydroxybenzophenone solution with an HPC concentration of 3 mass % and an 2,4-dihydroxybenzophenone concentration of 0.3 mass % was thus obtained. The HPC/2,4-dihydroxybenzophenone solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, an HPC/2,4-dihydroxybenzophenone nanofiber sheet with an average fiber outer diameter of approximately 0.2 to 2.0 μm was obtained. A nanofiber sheet was thus obtained with which, theoretically, 1% of 2,4-dihydroxybenzophenone is blended with respect to the mass of HPC.

### Example 24

### Preparation of HPC/Titanium Oxide Nanofibers

0.3 g of HPC (hydroxypropyl cellulose (H) made by Nippon Soda Co., Ltd.) and 9.7 g of ethanol (ethanol (no less than 99%, first grade, fermented) made by Japan Alcohol Trading Co., Ltd.) were placed and sealed in a sample vial, and complete dissolution was achieved by performing vibration stirring while warming. After dissolution of the HPC, 0.0009 g of titanium oxide (titanium oxide of bead form made by Wako Pure Chemical Industries, Ltd.) were added, and complete dissolution was achieved by further performing vibration stirring under room temperature. A collagen peptide/titanium oxide solution with a collagen peptide concentration of 3 mass % and a titanium oxide concentration of 0.009 mass % was thus obtained. The collagen peptide/titanium oxide solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a collagen peptide/titanium oxide nanofiber sheet with an average fiber outer diameter of approximately 0.2 to 2.2 μm was obtained. A nanofiber sheet was thus obtained with which, theoretically, 0.3% of titanium oxide is blended with respect to the mass of HPC.

### Example 25

### Preparation of HPC/Cerium Oxide Nanofibers

0.3 g of HPC (hydroxypropyl cellulose (H) made by Nippon Soda Co., Ltd.) and 9.7 g of ethanol (ethanol (no less than 99%, first grade, fermented) made by Japan Alcohol Trading Co., Ltd.) were placed and sealed in a sample vial, and complete dissolution was achieved by performing vibration stirring while warming. After dissolution of the HPC, 0.0009 g of cerium oxide (cerium (IV) oxide made by Wako Pure Chemical Industries, Ltd.) were added, and complete dissolution was achieved by further performing vibration stirring under room temperature. An HPC/cerium oxide solution with an HPC concentration of 45 mass % and a cerium oxide concentration of 0.009 mass % was thus obtained. The HPC/cerium oxide solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, an HPC/cerium oxide nanofiber sheet with an average fiber outer diameter of approximately 0.2 to 2.0 μm was obtained. A nanofiber sheet was thus obtained with which, theoretically, 0.3% of cerium oxide is blended with respect to the mass of HPC.

### Example 26

### Preparation of Collagen Peptide/Citric Acid Nanofibers

4.5 g of pig skin collagen peptide (collagen peptide PCH made by Unitec Foods Co., Ltd.) and 5.5 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. After dissolution of the collagen peptide, 0.045 g of citric acid (citric acid made by Wako Pure Chemical Industries, Ltd.) were added, and complete dissolution was achieved by further performing vibration stirring under room temperature. A collagen peptide/citric acid solution with a collagen peptide concentration of 45 mass % and a citric acid concentration of 0.45 mass % was thus obtained. The collagen peptide/citric acid solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a collagen peptide/citric acid nanofiber sheet with an average fiber outer diameter of approximately 0.1 to 2.0 μm was obtained. A nanofiber sheet was thus obtained with which, theoretically, 1% of citric acid is blended with respect to the mass of the collagen peptide.

### Example 27

### Preparation of Collagen Peptide/Succinic Acid Nanofibers

4.5 g of pig skin collagen peptide (collagen peptide PCH made by Unitec Foods Co., Ltd.) and 5.5 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. After dissolution of the collagen peptide, 0.045 g of succinic acid (succinic acid made by Wako Pure Chemical Industries, Ltd.) were added, and complete dissolution was achieved by further performing vibration stirring under room temperature. A collagen peptide/succinic acid solution with a collagen peptide concentration of 45 mass % and a succinic acid concentration of 0.45 mass % was thus obtained. The collagen peptide/succinic acid solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a collagen peptide/succinic acid nanofiber sheet with an average fiber outer diameter of approximately 0.1 to 2.0 μm was obtained. A nanofiber sheet was thus obtained with which, theoretically, 1% of succinic acid is blended with respect to the mass of the collagen peptide.

### Example 28

### Preparation of Collagen Peptide/Tea Leaf Extract Nanofibers

4.5 g of pig skin collagen peptide (collagen peptide PCH made by Unitec Foods Co., Ltd.) and 5.5 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. After dissolution of the collagen peptide, 0.045 g of a tea leaf extract (Camellia Extract 30S made by Taiyo Kagaku Co., Ltd.) were added, and complete dissolution was achieved by further performing vibration stirring under room temperature. A collagen peptide/tea leaf extract solution with a collagen peptide concentration of 45 mass % and a tea leaf extract concentration of 0.45 mass % was thus obtained. The collagen peptide/tea leaf extract solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a collagen peptide/tea leaf extract nanofiber sheet with an average fiber outer diameter of approximately 0.1 to 2.0 μm was obtained. A nanofiber sheet was thus obtained with which, theoretically, 1% of tea leaf extract is blended with respect to the mass of the collagen peptide.

### Example 29

### Preparation of HPC/Sulfur Nanofibers

0.3 g of HPC (hydroxypropyl cellulose (H) made by Nippon Soda Co., Ltd.) and 9.7 g of ethanol (ethanol (no less than 99%, first grade, fermented) made by Japan Alcohol Trading Co., Ltd.) were placed and sealed in a sample vial, and complete dissolution was achieved by performing vibration stirring while warming. After dissolution of the HPC, 0.0009 g of sulfur (sulfur made by Wako Pure Chemical Industries, Ltd.) were added, and complete dissolution was achieved by further performing vibration stirring under room temperature. An HPC/sulfur solution with an HPC concentration of 3 mass % and a ceric oxide concentration of 0.009 mass % was thus obtained. The HPC/sulfur solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, an HPC/sulfur nanofiber sheet with an average fiber outer diameter of approximately 0.3 to 1.8 μm was obtained. A nanofiber sheet was thus obtained with which, theoretically, 0.3% of sulfur is blended with respect to the mass of HPC.

### Example 30

### Preparation of PVA/Glycolic Acid Nanofibers

2.5 g of PVA (polyvinyl alcohol 3,500 made by Wako Pure Chemical Industries, Ltd.) and 7.5 g of ion-exchanged water were placed and sealed in a sample vial, and complete dissolution was achieved by performing vibration stirring while warming. After dissolution of the PVA, 0.025 g of glycolic acid (glycolic acid made by Wako Pure Chemical Industries, Ltd.) were added, and complete dissolution was achieved by further performing vibration stirring under room temperature. A PVA/glycolic acid solution with a PVA concentration of 25 mass % and a glycolic acid concentration of 0.25 mass % was thus obtained. The PVA/glycolic acid solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 6.

As a result, a PVA/glycolic acid nanofiber sheet with an average fiber outer diameter of approximately 0.1 to 2.5 μm was obtained. A nanofiber sheet was thus obtained with which, theoretically, 1% of glycolic acid is blended with respect to the mass of PVA.

### Example 31

### Preparation of HPC/Salicylic Acid Nanofibers

0.3 g of HPC (hydroxypropyl cellulose (H) made by Nippon Soda Co., Ltd.) and 9.7 g of ethanol (ethanol (no less than 99%, first grade, fermented) made by Japan Alcohol Trading Co., Ltd.) were placed and sealed in a sample vial, and complete dissolution was achieved by performing vibration stirring while warming. After dissolution of the HPC, 0.003 g of salicylic acid (salicylic acid made by Wako Pure Chemical Industries, Ltd.) were added, and complete dissolution was achieved by further performing vibration stirring under room temperature. An HPC/salicylic acid solution with an HPC concentration of 3 mass % and a salicylic acid concentration of 0.03 mass % was thus obtained. The HPC/salicylic acid solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, an HPC/salicylic acid nanofiber sheet with an average fiber outer diameter of approximately 0.3 to 1.8 μm was obtained. A nanofiber sheet was thus obtained with which, theoretically, 1% of salicylic acid is blended with respect to the mass of HPC.

### Example 32

### Preparation of Collagen Peptide/Licorice Extract Nanofibers

4.5 g of pig skin collagen peptide (collagen peptide PCH made by Unitec Foods Co., Ltd.) and 5.5 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. After dissolution of the collagen peptide, 0.045 g of a licorice extract (Licorice Extract No. 3 made by Takasago International Corporation) were added, and complete dissolution was achieved by further performing vibration stirring under room temperature. A collagen peptide/licorice extract solution with a collagen peptide concentration of 45 mass % and a licorice extract concentration of 0.45 mass % was thus obtained. The collagen peptide/licorice extract solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 1.

As a result, a collagen peptide/licorice extract nanofiber sheet with an average fiber outer diameter of approximately 0.4 to 2.8 μm was obtained. A nanofiber sheet was thus obtained with which, theoretically, 1% of licorice extract is blended with respect to the mass of the collagen peptide.

### Example 32

### Preparation of PVA/Allantoin Nanofibers

2.5 g of PVA (polyvinyl alcohol 3,500 made by Wako Pure Chemical Industries, Ltd.) and 7.5 g of ion-exchanged water were placed and sealed in a sample vial, and complete dissolution was achieved by performing vibration stirring while warming. After dissolution of the PVA, 0.025 g of allantoin (allantoin made by Wako Pure Chemical Industries, Ltd.) were added, and complete dissolution was achieved by further performing vibration stirring under room temperature. A PVA/allantoin solution with a PVA concentration of 25 mass % and an allantoin concentration of 0.25 mass % was thus obtained. The PVA/allantoin solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 6.

As a result, a PVA/allantoin nanofiber sheet with an average fiber outer diameter of approximately 0.1 to 2.5 μm was obtained. A nanofiber sheet was thus obtained with which, theoretically, 1% of allantoin is blended with respect to the mass of PVA.

### Example 33

### Manufacture of Antimicrobial Sterilizer

4.5 g of pig skin collagen peptide (collagen peptide PCH made by Unitec Foods Co., Ltd.) and 5.5 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. After dissolution of the pig skin collagen peptide, 0.045 g of benzethonium chloride (benzethonium chloride made by Wako Pure Chemical Industries, Ltd.), which is an antimicrobial substance, were added, and complete dissolution was achieved by further performing vibration stirring under room temperature. A pig skin collagen peptide/benzethonium chloride solution with a pig skin collagen peptide concentration of 45 mass % and a benzethonium chloride concentration of 0.45 mass % was thus obtained. The solution was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as in Example 6.

As a result, a nanofiber sheet (medical sheet) with an average fiber outer diameter of approximately 0.2 to 2.4 μm was obtained. A nanofiber sheet was thus obtained with which, theoretically, 1% of benzethonium chloride is blended with respect to the mass of the collagen peptide. This nanofiber sheet contains an antibacterial agent and dissolved rapidly in water.

After washing a suitable wound, the medical sheet was cut according to the size of the wound and just a necessary amount was adhered. The medical sheet dissolved instantaneously because it was adhered onto the wound in the wet state after washing and it was thus possible to apply the drug component without sensation of pain.

### Example 34 to Example 39

### Preparation of PVA/Collagen Peptide Nanofibers

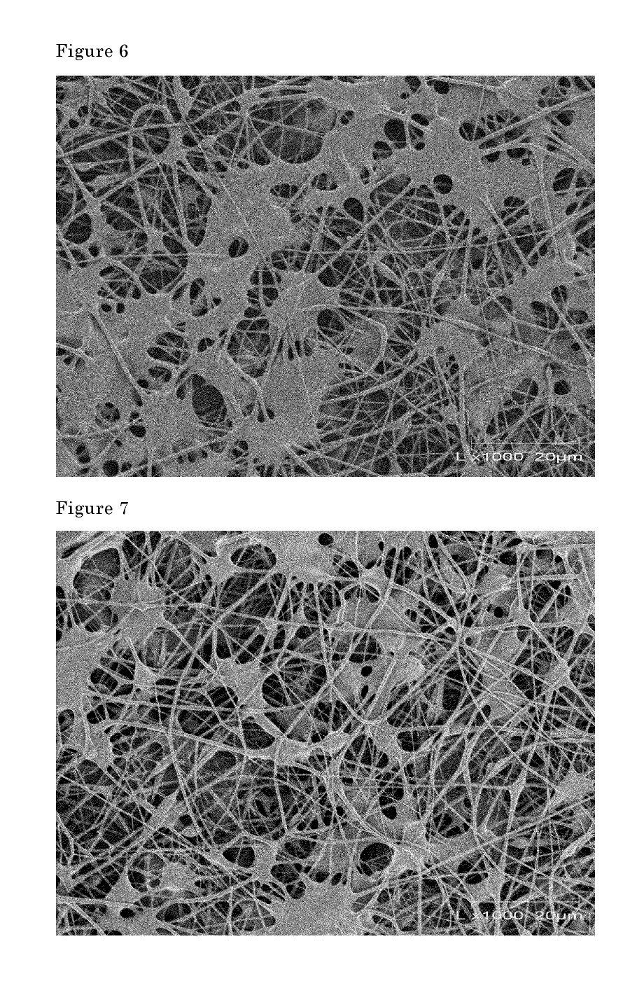

PVA (polyvinyl alcohol 3,500 made by Wako Pure Chemical Industries, Ltd.), collagen peptide (pig skin collagen peptide PCH made by Unitec Foods Co., Ltd.), and 50 w/w % ethanol (ethanol (no less than 99%, first grade, fermented) made by Japan Alcohol Trading Co., Ltd.; ethanol:water mass ratio=50:50) were placed and sealed in sample vials, and complete dissolution was achieved by performing vibration stirring while warming. PVA/collagen peptide solutions were obtained by setting the mass ratios of the PVA, collagen peptide, and 50 w/w % ethanol as shown in Table 3 and formulating to attain a total mass of 10.00 g. Each PVA/collagen peptide solution obtained was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the spinning conditions shown below. As a result, with all examples, a PVA/collagen peptide nanofiber sheet with an average fiber outer diameter of approximately 0.1 to 2.5 μm was obtained. Electron micrographs are shown in FIG. 6 to FIG. 11.

Spinning conditions

| | |
|---|---|
| Voltage | 20 kV |
| Ejection rate | 2 ml/hr |
| Ejection distance | 15 cm |
| Temperature inside apparatus | 20 to 25° C. |
| Humidity inside apparatus | no more than 50% |

TABLE 3

| | Weight ratio | | Collagen | |
|---|---|---|---|---|
| | (PVA:CP:50 wt % | PVA | peptide | 50 wt % |
| Example No. | EtOH) | (g) | (CP) (g) | EtOH |
| 34 | 100:40:2000 | 0.467 | 0.187 | 9.346 |
| 35 | 100:50:2000 | 0.465 | 0.233 | 9.302 |
| 36 | 100:60:2000 | 0.463 | 0.278 | 9.259 |
| 37 | 100:80:2000 | 0.459 | 0.367 | 9.174 |
| 38 | 100:100:2000 | 0.455 | 0.455 | 9.090 |
| 39 | 100:150:2000 | 0.444 | 0.667 | 8.889 |

Also, the solubilities and handling properties as skin adhesion sheets of the respective PVA/collagen peptide nanofiber sheets obtained were evaluated, and the results are shown in Table 4. Example 1 and Example 6 were used as comparison controls.

### <Evaluation Standards of Solubility>

⊚: Dissolves instantaneously when floated on water.
∘: Dissolves after several seconds when floated on water.
⊚: Dissolves after several minutes when floated on water.
×: Does not dissolve even when floated on water.

### <Evaluation Standards of Handling Property>

⊚: Does not break apart or dissolve at all even when touched with the hand and can be adhered readily onto skin.
∘: Although breaking apart or dissolving slightly when touched with the hand, can be adhered readily onto skin.
⊚: Breaks apart or dissolves considerably when touched with the hand and is difficult to adhere onto skin.
×: Breaks apart or dissolves completely and instantaneously when touched with the hand and cannot be adhered onto skin.

TABLE 4

| | Example No. | | | | | | | |
|---|---|---|---|---|---|---|---|---|
| | 1 | 6 | 34 | 35 | 36 | 37 | 38 | 39 |
| Solubility | ⊚ | Δ | Δ | ⊚ | ⊚ | ⊚ | ◯ | X |
| Handling | X | ⊚ | ◯ | ◯ | ⊚ | ◯ | Δ | Δ |
| property | | | | | | | | |
| Comprehensive | Δ | ◯ | ◯ | ⊚ | ⊚ | ⊚ | ◯ | Δ |
| evaluation | | | | | | | | |

### Example 40 to Example 46

### Preparation of PEG/Collagen Peptide Nanofibers

PEG (polyethylene glycol 500,000 made by Wako Pure Chemical Industries, Ltd.), collagen peptide (pig skin collagen peptide PCH made by Unitec Foods Co., Ltd.), and 50 w/w % ethanol (ethanol (no less than 99%, first grade, fermented) made by Japan Alcohol Trading Co., Ltd.; ethanol:water mass ratio=50:50) were placed and sealed in sample vials, and complete dissolution was achieved by performing vibration stirring while warming. PEG/collagen peptide solutions were obtained by setting the mass ratios of the PEG, collagen peptide, and 50 w/w % ethanol as shown in Table 5 and formulating to attain a total mass of 10.00 g. Each PEG/collagen peptide solution obtained was loaded into a syringe (made by Terumo Corp.), a 23 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as Example 34 to Example 39. As a result, with all examples, a PEG/collagen peptide nanofiber sheet with an average fiber outer diameter of approximately 0.2 to 2.2 μm was obtained.

TABLE 5

| | Weight ratio | | Collagen | |
|---|---|---|---|---|
| Example | (PEG:CP:50 wt % | PEG | peptide | 50 wtEtOH |
| No. | EtOH) | (g) | (CP) (g) | (g) |
| 40 | 0.1:45:55 | 0.010 | 4.496 | 5.494 |
| 41 | 0.5:45:55 | 0.050 | 4.478 | 5.472 |
| 42 | 1.0:45:55 | 0.099 | 4.455 | 5.446 |
| 43 | 1.5:45:55 | 0.148 | 4.433 | 5.419 |
| 44 | 2.0:45:55 | 0.196 | 4.412 | 5.392 |
| 45 | 2.5:45:55 | 0.244 | 4.390 | 5.366 |
| 46 | 2.0:0:98 | 0.200 | 0.000 | 9.800 |

Also, the solubilities and handling properties as skin adhesion sheets of the respective PEG/collagen peptide nanofiber sheets obtained were evaluated, and the results are shown in Table 6. Example 1 was used as a comparison control.

TABLE 6

| | Example No. | | | | | | | |
|---|---|---|---|---|---|---|---|---|
| | 1 | 40 | 41 | 42 | 43 | 44 | 45 | 46 |
| Solubility | X | ◯ | ⊚ | ⊚ | ◯ | ◯ | Δ | Δ |
| Handling | ⊚ | Δ | ◯ | ⊚ | ⊚ | ◯ | ◯ | Δ |
| property | | | | | | | | |
| Comprehensive | Δ | ◯ | ⊚ | ⊚ | ⊚ | ◯ | ◯ | Δ |
| evaluation | | | | | | | | |

### Example 47 to Example 52

### Preparation of HPC/Quince Seed Gum Nanofibers

HPC (hydroxypropyl cellulose (H) made by Nippon Soda Co., Ltd.), quince seed gum (quince seed powder made by Taiyo Kagaku Co., Ltd.), and ethanol (ethanol (no less than 99%, first grade, fermented) made by Japan Alcohol Trading Co., Ltd.) were placed and sealed in sample vials, and complete dissolution was achieved by performing vibration stirring while warming. HPC/quince seed gum solutions were obtained by setting the mass ratios of the HPC, quince seed gum, and ethanol as shown in Table 7 and formulating to attain a total mass of 10.00 g. Each HPC/quince seed gum solution obtained was loaded into a syringe (made by Terumo Corp.), a 21 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as Example 34 to Example 39. As a result, with all examples, an HPC/quince seed gum nanofiber sheet with an average fiber outer diameter of approximately 0.2 to 2.0 μm was obtained.

TABLE 7

| | | | Quince seed | |
|---|---|---|---|---|
| | Weight ratio | HPC | gum (QSG) | EtOH |
| Example No. | (HPC:QSG:EtOH) | (g) | (g) | (g) |
| 47 | 0.1:3:97 | 0.010 | 0.300 | 9.690 |
| 48 | 0.5:3:97 | 0.050 | 0.299 | 9.651 |
| 49 | 1.0:3:97 | 0.099 | 0.297 | 9.604 |
| 50 | 1.5:3:97 | 0.148 | 0.296 | 9.557 |
| 51 | 2.0:3:97 | 0.244 | 0.293 | 9.463 |
| 52 | 2.5:3:97 | 0.291 | 0.291 | 9.418 |

Also, the solubilities and handling properties as skin adhesion sheets of the respective HPC/quince seed gum nanofiber sheets obtained were evaluated, and the results are shown in Table 8. Example 3 and Example 5 were used as comparison controls.

TABLE 8

| | Example No. | | | | | | | |
|---|---|---|---|---|---|---|---|---|
| | 3 | 5 | 47 | 48 | 49 | 50 | 51 | 52 |
| Solubility | Δ | ⊚ | ⊚ | ⊚ | ⊚ | ⊚ | ◯ | ◯ |
| Handling | ⊚ | X | Δ | ◯ | ⊚ | ⊚ | ⊚ | Δ |
| property | | | | | | | | |
| Comprehensive | ◯ | Δ | ◯ | ⊚ | ⊚ | ⊚ | ⊚ | ◯ |
| evaluation | | | | | | | | |

### Example 53 to Example 58

### Preparation of Sodium Polyacrylate/Quince Seed Gum Nanofibers

Sodium polyacrylate (Viscomate made by Showa Denko K. K.), quince seed gum (quince seed powder made by Taiyo Kagaku Co., Ltd.), and 50 w/w % ethanol (ethanol (no less than 99%, first grade, fermented) made by Japan Alcohol Trading Co., Ltd.; ethanol:water mass ratio=50:50) were placed and sealed in sample vials, and complete dissolution was achieved by performing vibration stirring while warming. Sodium polyacrylate/quince seed gum solutions were obtained by setting the mass ratios of the sodium polyacrylate, quince seed gum, and 50 w/w % ethanol as shown in Table 9 and formulating to attain a total mass of 10.00 g. Each sodium polyacrylate/quince seed gum solution obtained was loaded into a syringe (made by Terumo Corp.), a 21 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as Example 34 to Example 39. As a result, with all examples, a sodium polyacrylate/quince seed gum nanofiber sheet with an average fiber outer diameter of approximately 0.2 to 2.2 μm was obtained.

TABLE 9

| | Weight ratio | Sodium | Quince seed | 50 wt % |
|---|---|---|---|---|
| | (PA-Na:QSG:50 | polyacrylate | gum (QSG) | EtOH |
| Example No. | wt % EtOH) | (PA-Na) (g) | (g) | (g) |
| 53 | 0.1:3:97 | 0.010 | 0.300 | 9.690 |
| 54 | 0.3:3:97 | 0.030 | 0.299 | 9.671 |
| 55 | 0.5:3:97 | 0.050 | 0.299 | 9.651 |
| 56 | 0.7:3:97 | 0.070 | 0.298 | 9.632 |
| 57 | 1.0:3:97 | 0.099 | 0.297 | 9.604 |
| 58 | 1.2:3:97 | 0.119 | 0.298 | 9.585 |

Also, the solubilities and handling properties as skin adhesion sheets of the respective sodium polyacrylate/quince seed gum nanofiber sheets obtained were evaluated, and the results are shown in Table 10. Example 5 and Example 7 were used as comparison controls.

TABLE 10

| | Example No. | | | | | | | |
|---|---|---|---|---|---|---|---|---|
| | 5 | 7 | 53 | 54 | 55 | 56 | 57 | 58 |
| Solubility | ⊚ | ◯ | ⊚ | ⊚ | ⊚ | ⊚ | ⊚ | ◯ |
| Handling | X | ◯ | Δ | Δ | ◯ | ◯ | ◯ | ◯ |
| property | | | | | | | | |
| Comprehensive | Δ | ◯ | ◯ | ◯ | ⊚ | ⊚ | ⊚ | ◯ |
| evaluation | | | | | | | | |

### Example 59 to Example 64

### Preparation of PEG/Silk Fibroin Nanofibers

PEG (polyethylene glycol 500,000 made by Wako Pure Chemical Industries, Ltd.), silk fibroin (silk fibroin made by Silk Kogei K. K.), and 30 w/w % ethanol (ethanol (no less than 99%, first grade, fermented) made by Japan Alcohol Trading Co., Ltd.; ethanol:water mass ratio=30:70) were placed and sealed in sample vials, and complete dissolution was achieved by performing vibration stirring while warming. PEG/silk fibroin solutions were obtained by setting the mass ratios of the PEG, silk fibroin, and 30 w/w % ethanol as shown in Table 11 and formulating to attain a total mass of 10.00 g. Each PEG/silk fibroin solution obtained was loaded into a syringe (made by Terumo Corp.), a 21 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as Example 34 to Example 39. As a result, with all examples, a PEG/silk fibroin nanofiber sheet with an average fiber outer diameter of approximately 0.2 to 2.2 μm was obtained.

TABLE 11

| | Weight ratio | | Silk | |
|---|---|---|---|---|
| | (PEG:SF:30 wt % | PEG | fibroin | 30 wt % EtOH |
| Example No. | EtOH) | (g) | (SF) (g) | (g) |
| 59 | 0.1:45:55 | 0.010 | 4.496 | 5.494 |
| 60 | 0.5:45:55 | 0.050 | 4.478 | 5.472 |
| 61 | 1.0:45:55 | 0.099 | 4.455 | 5.446 |
| 62 | 1.5:45:55 | 0.148 | 4.433 | 5.419 |
| 63 | 2.0:45:55 | 0.196 | 4.412 | 5.392 |
| 64 | 2.5:45:55 | 0.244 | 4.390 | 5.366 |

Also, the solubilities and handling properties as skin adhesion sheets of the respective PEG/silk fibroin nanofiber sheets obtained were evaluated, and the results are shown in Table 12. Example 8 and Example 46 were used as comparison controls.

TABLE 12

| | Example No. | | | | | | | |
|---|---|---|---|---|---|---|---|---|
| | 8 | 46 | 59 | 60 | 61 | 62 | 63 | 64 |
| Solubility | ⊚ | Δ | ⊚ | ⊚ | ⊚ | ⊚ | ⊚ | ◯ |
| Handling | Δ | Δ | Δ | Δ | Δ | ◯ | ⊚ | ⊚ |
| property | | | | | | | | |
| Comprehensive | ◯ | Δ | ◯ | ◯ | ◯ | ⊚ | ⊚ | ⊚ |
| evaluation | | | | | | | | |

### Example 65 to Example 70

### Preparation of Sodium Polyacrylate/Silk Fibroin Nanofibers

Sodium polyacrylate (Viscomate made by Showa Denko K. K.), silk fibroin (silk fibroin made by Silk Kogei K. K.), and 30 w/w % ethanol (ethanol (no less than 99%, first grade, fermented) made by Japan Alcohol Trading Co., Ltd.; ethanol:water mass ratio=30:70) were placed and sealed in sample vials, and complete dissolution was achieved by performing vibration stirring while warming. Sodium polyacrylate/silk fibroin solutions were obtained by setting the mass ratios of the sodium polyacrylate, silk fibroin, and 30 w/w % ethanol as shown in Table 13 and formulating to attain a total mass of 10.00 g. Each sodium polyacrylate/silk fibroin solution obtained was loaded into a syringe (made by Terumo Corp.), a 21 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as Example 34 to Example 39. As a result, with all examples, a sodium polyacrylate/silk fibroin nanofiber sheet with an average fiber outer diameter of approximately 0.3 to 2.5 μm was obtained.

TABLE 13

| | Weight ratio | Sodium | Silk | 30 wt % |
|---|---|---|---|---|
| Example | (PA-Na:SF:30 wt % | polyacrylate | fibroin | EtOH |
| No. | EtOH) | (PA-Na) (g) | (SF) (g) | (g) |
| 65 | 0.1:45:55 | 0.010 | 4.496 | 5.494 |
| 66 | 0.5:45:55 | 0.050 | 4.478 | 5.472 |
| 67 | 1.0:45:55 | 0.099 | 4.455 | 5.446 |
| 68 | 1.5:45:55 | 0.148 | 4.433 | 5.419 |
| 69 | 2.0:45:55 | 0.196 | 4.412 | 5.392 |
| 70 | 2.5:45:55 | 0.244 | 4.390 | 5.366 |

Also, the solubilities and handling properties as skin adhesion sheets of the respective sodium polyacrylate/silk fibroin nanofiber sheets obtained were evaluated, and the results are shown in Table 14. Example 7 and Example 8 were used as comparison controls.

TABLE 14

| | Example No. | | | | | | | |
|---|---|---|---|---|---|---|---|---|
| | 7 | 8 | 65 | 66 | 67 | 68 | 69 | 70 |
| Solubility | ◯ | ⊚ | ◯ | ◯ | ⊚ | ⊚ | ⊚ | ⊚ |
| Handling | ◯ | Δ | Δ | Δ | ◯ | ⊚ | ◯ | ◯ |
| property | | | | | | | | |
| Comprehensive | ◯ | ◯ | ◯ | ◯ | ⊚ | ⊚ | ⊚ | ⊚ |
| evaluation | | | | | | | | |

### Example 71 to Example 76

### Preparation of PVA/Gelatin Nanofibers

PVA (polyvinyl alcohol 3,500 made by Wako Pure Chemical Industries, Ltd.), gelatin (Neosoft GE-388 made by Taiyo Kagaku Co., Ltd.), and 30 w/w % ethanol (ethanol (no less than 99%, first grade, fermented) made by Japan Alcohol Trading Co., Ltd.; ethanol:water mass ratio=30:70) were placed and sealed in sample vials, and complete dissolution was achieved by performing vibration stirring while warming. PVA/gelatin solutions were obtained by setting the mass ratios of the PVA, gelatin, and 30 w/w % ethanol as shown in Table 15 and formulating to attain a total mass of 10.00 g. Each PVA/gelatin solution obtained was loaded into a syringe (made by Terumo Corp.), a 21 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as Example 34 to Example 39. As a result, with all examples, a PVA/gelatin nanofiber sheet with an average fiber outer diameter of approximately 0.15 to 2.0 μm was obtained.

TABLE 15

| | Weight ratio | | | |
|---|---|---|---|---|
| | (PVA:gelatin:30 wt % | PVA | | 30 wt % EtOH |
| Example No. | EtOH) | (g) | Gelatin (g) | (g) |
| 71 | 0.1:12.5:87.5 | 0.010 | 1.249 | 8.741 |
| 72 | 0.25:12.5:87.5 | 0.025 | 1.247 | 8.728 |
| 73 | 0.5:12.5:87.5 | 0.050 | 1.244 | 8.706 |
| 74 | 1.0:12.5:87.5 | 0.099 | 1.238 | 8.663 |
| 75 | 1.5:12.5:87.5 | 0.148 | 1.232 | 8.620 |
| 76 | 2.0:12.5:87.5 | 0.196 | 1.225 | 8.578 |

Also, the solubilities and handling properties as skin adhesion sheets of the respective PVA/gelatin nanofiber sheets obtained were evaluated, and the results are shown in Table 16. Example 2 and Example 6 were used as comparison controls.

TABLE 16

| | Example No. | | | | | | | |
|---|---|---|---|---|---|---|---|---|
| | 2 | 6 | 71 | 72 | 73 | 74 | 75 | 76 |
| Solubility | ◯ | Δ | ⊚ | ⊚ | ⊚ | ⊚ | ⊚ | ◯ |
| Handling | Δ | ⊚ | Δ | Δ | ◯ | ⊚ | ⊚ | ⊚ |
| property | | | | | | | | |
| Comprehensive | ◯ | ◯ | ◯ | ◯ | ⊚ | ⊚ | ⊚ | ⊚ |
| evaluation | | | | | | | | |

### Example 77 to Example 82

### Preparation of HPC/Gelatin Nanofibers

HPC (hydroxypropyl cellulose (H) made by Nippon Soda Co., Ltd.), gelatin (Neosoft GE-388 made by Taiyo Kagaku Co., Ltd.), and 30 w/w % ethanol (ethanol (no less than 99%, first grade, fermented) made by Japan Alcohol Trading Co., Ltd.; ethanol:water mass ratio=30:70) were placed and sealed in sample vials, and complete dissolution was achieved by performing vibration stirring while warming. HPC/gelatin solutions were thus obtained by setting the mass ratios of the HPC, gelatin, and 30 w/w % ethanol as shown in Table 17 and formulating to attain a total mass of 10.00 g. Each HPC/gelatin solution obtained was loaded into a syringe (made by Terumo Corp.), a 21 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as Example 34 to Example 39. As a result, with all examples, an HPC/gelatin nanofiber sheet with an average fiber outer diameter of approximately 0.20 to 2.0 μm was obtained.

TABLE 17

| | Weight ratio | | | |
|---|---|---|---|---|
| | (HPC:gelatin:30 wt % | HPC | | 30 wt % EtOH |
| Example No. | EtOH) | (g) | Gelatin (g) | (g) |
| 77 | 0.1:12.5:87.5 | 0.010 | 1.249 | 8.741 |
| 78 | 0.25:12.5:87.5 | 0.025 | 1.247 | 8.728 |
| 79 | 0.5:12.5:87.5 | 0.050 | 1.244 | 8.706 |
| 80 | 1.0:12.5:87.5 | 0.099 | 1.238 | 8.663 |
| 81 | 1.5:12.5:87.5 | 0.148 | 1.232 | 8.620 |
| 82 | 2.0:12.5:87.5 | 0.196 | 1.225 | 8.578 |

Also, the solubilities and handling properties as skin adhesion sheets of the respective HPC/gelatin nanofiber sheets obtained were evaluated, and the results are shown in Table 18. Example 2 and Example 3 were used as comparison controls.

TABLE 18

| | Example No. | | | | | | | |
|---|---|---|---|---|---|---|---|---|
| | 2 | 3 | 77 | 78 | 79 | 80 | 81 | 82 |
| Solubility | ⊚ | Δ | ◯ | ⊚ | ⊚ | ⊚ | ◯ | ◯ |
| Handling | Δ | ⊚ | Δ | Δ | ◯ | ⊚ | ⊚ | ◯ |
| property | | | | | | | | |
| Comprehensive | ◯ | ◯ | ◯ | ◯ | ⊚ | ⊚ | ⊚ | ◯ |
| evaluation | | | | | | | | |

### Example 83

### Preparation of PVA/Collagen Peptide/Theanine Nanofibers

0.463 g of PVA (polyvinyl alcohol 3,500 made by Wako Pure Chemical Industries, Ltd.), 0.278 g of collagen peptide (pig skin collagen peptide PCH made by Unitec Foods Co., Ltd.), 0.0463 g of theanine (Suntheanine made by Taiyo Kagaku Co., Ltd.), and 9.259 g of 50 w/w % ethanol (ethanol (no less than 99%, first grade, fermented) made by Japan Alcohol Trading Co., Ltd.; ethanol:water mass ratio=50:50) were placed and sealed in a sample vial, and complete dissolution was achieved by performing vibration stirring while warming. The PVA/collagen peptide/theanine solution was loaded into a syringe (made by Terumo Corp.), a 21 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as Example 34 to Example 39. As a result, a PVA/collagen peptide/theanine nanofiber sheet with an average fiber outer diameter of approximately 0.1 to 2.0 μm was obtained. A nanofiber sheet with a theoretical mass ratio of PVA:collagen peptide:theanine=100:60:1 was thus obtained.

### Example 84

### Preparation of HPC/Quince Seed Gum/CoQ10 Nanofibers

0.099 g of HPC (hydroxypropyl cellulose (H) made by Nippon Soda Co., Ltd.), 0.297 g of quince seed gum (quince seed powder made by Taiyo Kagaku Co., Ltd.), 0.0099 g of a CoQ10 formulation (SUN ACTIVE Q-10Y, with a CoQ10 concentration of 10 mass %; made by Taiyo Kagaku Co., Ltd.), and 9.604 g of ethanol (ethanol (no less than 99%, first grade, fermented) made by Japan Alcohol Trading Co., Ltd.) were placed and sealed in a sample vial, and complete dissolution was achieved by performing vibration stirring while warming. The HPC/quince seed gum/CoQ10 solution was loaded into a syringe (made by Terumo Corp.), a 21 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as Example 34 to Example 39. As a result, an HPC/quince seed gum/CoQ10 nanofiber sheet with an average fiber outer diameter of approximately 0.20 to 2.0 μm was obtained. A nanofiber sheet with a theoretical mass ratio of HPC:quince seed gum:CoQ10=100:300:1 was thus obtained.

### Example 85

### Preparation of PEG/Silk Fibroin/Vitamin C Nanofibers

0.196 g of PEG (polyethylene glycol 500,000 made by Wako Pure Chemical Industries, Ltd.), 4.412 g of silk fibroin (silk fibroin made by Silk Kogei K. K.), 0.0099 g of vitamin C (sodium ascorbate made by Tanabe Pharma Corp.), and 5.392 g of 30 w/w % ethanol (ethanol (no less than 99%, first grade, fermented) made by Japan Alcohol Trading Co., Ltd.; ethanol:water mass ratio=30:70) were placed and sealed in a sample vial, and complete dissolution was achieved by performing vibration stirring while warming. The PEG/silk fibroin/vitamin C solution was loaded into a syringe (made by Terumo Corp.), a 21 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as Example 34 to Example 39. As a result, a PEG/silk fibroin/vitamin C nanofiber sheet with an average fiber outer diameter of approximately 0.20 to 2.1 μm was obtained. A nanofiber sheet with a theoretical mass ratio of PEG:silk fibroin:vitamin C=4.4:100:5 was thus obtained.

### Example 86

### Preparation of HPC/Gelatin/Sodium Hyaluronate Nanofibers

0.099 g of HPC (hydroxypropyl cellulose (H) made by Nippon Soda Co., Ltd.), 1.238 g of gelatin (Neosoft GE-388 made by Taiyo Kagaku Co., Ltd.), 0.001238 g of sodium hyaluronate (sodium hyaluronate made by Wako Pure Chemical Industries, Ltd.), and 8.663 g of 30 w/w % ethanol (ethanol (no less than 99%, first grade, fermented) made by Japan Alcohol Trading Co., Ltd.; ethanol:water mass ratio=30:70) were placed and sealed in a sample vial, and complete dissolution was achieved by performing vibration stirring while warming. The HPC/gelatin/sodium hyaluronate solution was loaded into a syringe (made by Terumo Corp.), a 21 G needle (made by Hoshiseido Medical Instrumentation Co., Ltd.) was attached to the tip of the syringe, and air bubbles inside the syringe were removed completely. The syringe was set on a syringe pump of an electrospinning apparatus (made by Imoto Machinery Co., Ltd.), and spinning was carried out under the same spinning conditions as Example 34 to Example 39. As a result, an HPC/gelatin/sodium hyaluronate nanofiber sheet with an average fiber outer diameter of approximately 0.20 to 2.0 μm was obtained. A nanofiber sheet with a theoretical mass ratio of HPC:gelatin:sodium hyaluronate=8:100:0.1 was thus obtained.

As described above, by the present embodiments, water-soluble electrospun sheets can be provided using predetermined base materials. These sheets dissolve in water readily and can thus be used as various materials, such as cosmetic sheets (including cosmetic facial masks, cosmetic toners, and beauty serums), medical sheets, etc.

Also by blending two types of base material, water-soluble electrospun sheets with which the handling property is enhanced over using just one type of base material can be provided while maintaining the immediately dissolving property.

Also, by making another functional component (for example, a humectant component, skin-whitening component, anti-ultraviolet component, astringent component, keratin-softening component, anti-inflammatory component, coloring component, etc.,) be contained in addition to the base material, the solution resulting from dissolving the sheet can be made to exhibit a specific function at a portion at which the sheet is adhered or at a portion at which the solution is rubbed in after dissolution of the sheet.

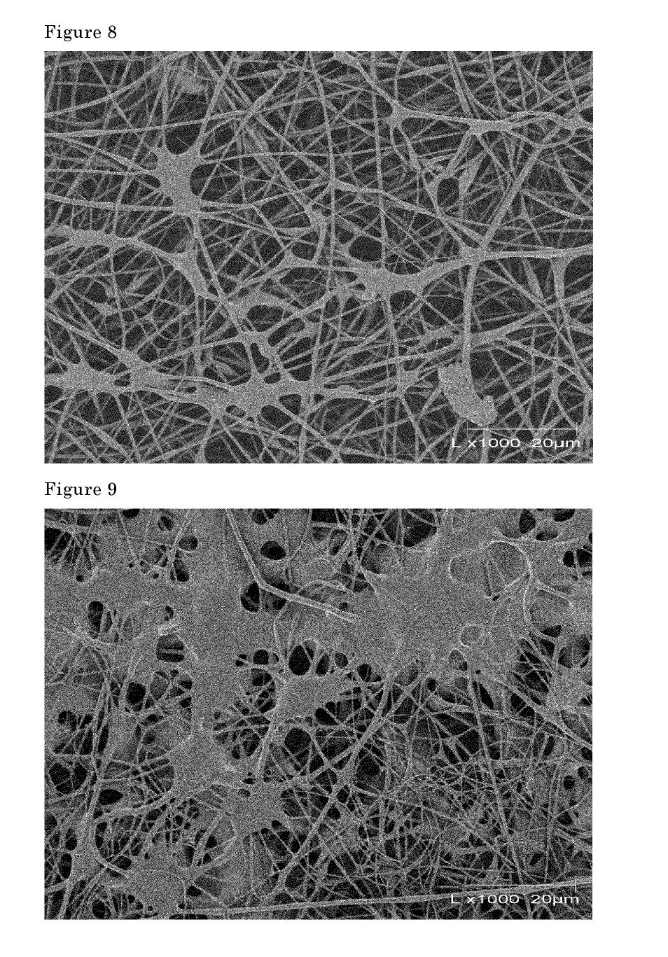

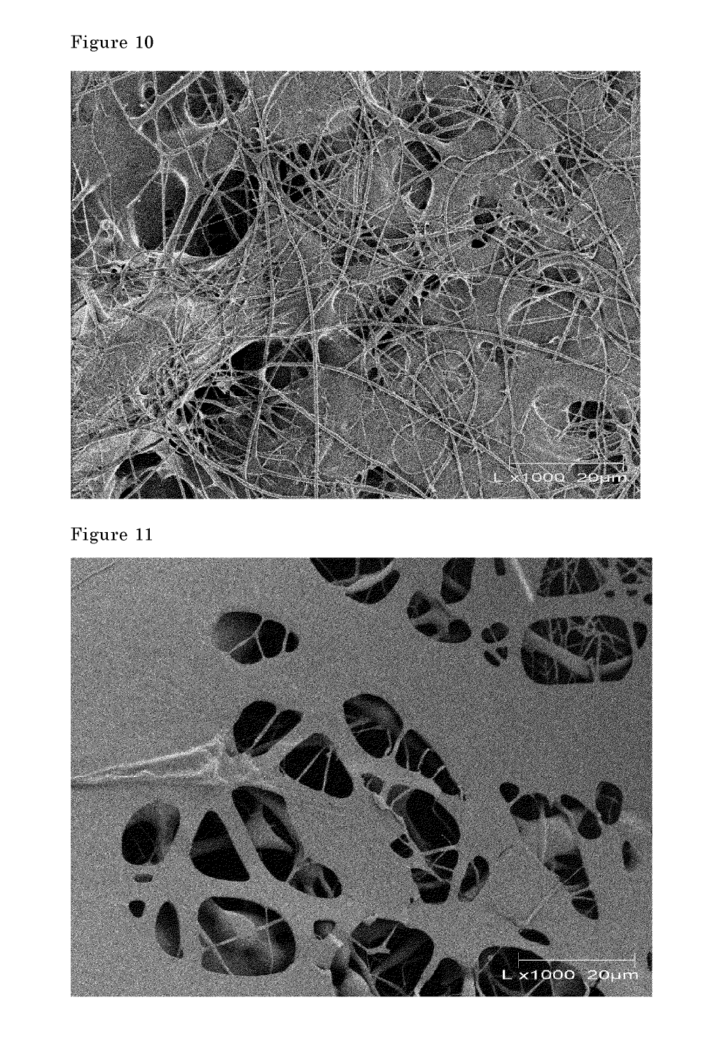

### BRIEF DESCRIPTION OF THE DRAWINGS

[FIG. 1] is a diagram for describing an electrospinning method in outline.

[FIG. 2] is an electron micrograph of a water-soluble electrospun sheet of Example 1 (magnification: 2000 times).

[FIG. 3] is an electron micrograph of a water-soluble electrospun sheet of Example 2 (magnification: 5000 times).

[FIG. 4] is an electron micrograph of a water-soluble electrospun sheet of Example 3 (magnification: 2000 times).

[FIG. 5] is an electron micrograph of a water-soluble electrospun sheet of Example 4 (magnification: 1000 times).

[FIG. 6] is an electron micrograph of a water-soluble electrospun sheet of Example 34 (magnification: 1000 times).

[FIG. 7] is an electron micrograph of a water-soluble electrospun sheet of Example 35 (magnification: 1000 times).

[FIG. 8] is an electron micrograph of a water-soluble electrospun sheet of Example 36 (magnification: 1000 times).

[FIG. 9] is an electron micrograph of a water-soluble electrospun sheet of Example 37 (magnification: 1000 times).

[FIG. 10] is an electron micrograph of a water-soluble electrospun sheet of Example 38 (magnification: 1000 times).

[FIG. 11] is an electron micrograph of a water-soluble electrospun sheet of Example 39 (magnification: 1000 times).

1. A water-soluble electrospun sheet comprising
a water-soluble base material.

2. The water-soluble electrospun sheet according to claim 1, wherein
the water-soluble base material is at least one material selected from a group consisting of: high-molecular proteins and decomposition products thereof; cellulose-based polymers; plant-based polymers and decomposition products thereof; vinyl-based polymers; acrylic-based polymers; and water-soluble polysaccharides.

3. The water-soluble electrospun sheet according to claim 1, wherein
the water-soluble base material is at least one material selected from a group consisting of: collagen peptide; gelatin, silk fibroin; hydroxypropyl cellulose; quince seed gum; hyaluronic acid; polyvinyl alcohol; sodium polyacrylate; and water-soluble chitosan.

4. The water-soluble electrospun sheet according to claim 1, further comprising
at least one functional component selected from among: emulsifying components;
stabilizing components; antimicrobial components; humectant components; skin-whitening components; anti-ultraviolet components; astringent components; keratin-softening components; anti-inflammatory components; emollient components; and
coloring components.

5. The water-soluble electrospun sheet according to claim 4, wherein
the functional component is at least one component selected from a group consisting of: theanine; hyaluronic acid; vitamin C; CoQ10; urea; hydrolyzed eggshell membrane; sodium chondroitin sulfate; glycol salicylate; diphenhydramine hydrochloride; salicylic acid; arbutin; citric acid; succinic acid; tea leaf extract; licorice extract; glycolic acid; allantoin; glycerin; 1,3-butylene glycol; ellagic acid; 2,4-dihydroxybenzophenone;
titanium oxide; cerium oxide; and sulfur.

6. The water-soluble electrospun sheet according to claim 1, wherein
the water-soluble electrospun sheet is a cosmetic sheet.

7. The water-soluble electrospun sheet according to claim 6, wherein the cosmetic sheet is a cosmetic facial mask, a cosmetic toner, or a beauty serum.

8. The water-soluble electrospun sheet according to claim 1, wherein
the water-soluble electrospun sheet is a medical sheet.

9. The water-soluble electrospun sheet according to claim 8, wherein
the medical sheet contains an antimicrobial substance or an anti-inflammatory substance.
